(19) United States
(12) Patent Application Publication
(10) Pub. No.: US 2022/0287969 A1
(43) Pub. Date: Sep. 15, 2022

(54) MULTILAMELLAR RNA NANOPARTICLES

(71) Applicant: University of Florida Research Foundation, Inc., Gainesville, FL (US)

(72) Inventors: Elias Sayour, Newberry, FL (US); Hector Ruben Mendez-Gomez, Gainesville, FL (US); Duane Mitchell, Gainesville, FL (US); Carlos Rinaldi, Gainesville, FL (US)

(21) Appl. No.: 17/626,674

(22) PCT Filed: Jul. 17, 2020
(86) PCT No.: PCT/US20/42606

§ 371 Date: Jan. 12, 2022

Related U.S. Application Data
(60) Provisional application No. 62/933,326, filed on Nov. 8, 2019.
(60) Provisional application No. 62/884,983, filed on Aug. 9, 2019.
(60) Provisional application No. 62/877,598, filed on Jul. 23, 2019.
(60) Provisional application No. 62/876,440, filed on Jul. 19, 2019.

(51) Int. Cl. A61K 9/127; C12N 15/113; C12N 15/88; A61K 39/39; A61K 48/00

(52) U.S. Cl. CPC A61K 9/1272; C12N 15/113; C12N 15/88; A61K 39/39; A61K 48/0033; C12N 2310/351; A61K 2039/53

(57) ABSTRACT

The present disclosure provides a nanoparticle comprising a positively-charged surface and an interior comprising (i) a core and (ii) at least two nucleic acid layers, wherein each nucleic acid layer is positioned between a cationic lipid bilayer. Methods of making such nanoparticles are further provided herein. Additionally, related cells, populations of cells, pharmaceutical compositions comprising the presently disclosed nanoparticles are provided. Methods of increasing an immune response against a tumor in a subject, methods of delivering RNA molecules to an intra-tumoral microenvironment, lymph node, and/or a reticuloendothelial organ in a subject, and methods of treating a subject with a disease are furthermore provided.

### CROSS-REFERENCE TO RELATED APPLICATIONS

This application claims the benefit of priority of U.S. Provisional Patent Application No. 62/876,440, filed Jul. 19, 2019, U.S. Provisional Patent Application No. 62/877,598, filed Jul. 23, 2019, U.S. Provisional Patent Application No. 62/884,983, filed Aug. 9, 2019, and U.S. Provisional Patent Application No. 62/933,326, filed Nov. 8, 2019, the disclosures of which are hereby incorporated by reference in their entirety;

This invention was made with government support under grant number K08 CA199224 awarded by The National Institutes of Health, and under grant number W81XWH-17-1-0510 awarded by the U.S. Army Medical Research Acquisition. The government has certain rights in the invention.

### BACKGROUND

Due to severe and non-specific deleterious effects of radiation and chemotherapy, targeted therapies capable of selectively killing tumor cells in patients with glioblastoma (GBM) are essential (Stupp et al., The New England Journal of Medicine. 2005; 352(10):987-96; Stupp et al., The Lancet Oncology. 2009; 10(5):459-66; Sampson et al., Journal of Clinical Oncology: Official Journal of the American Society of Clinical Oncology. 2010; 28(31):4722-9). Tumor-specific immunotherapy can be harnessed to eradicate malignant brain tumors with exquisite precision and without collateral damage to normal tissue (Schuster et al., Journal of clinical oncology: Official Journal of the American Society of Clinical Oncology. 2011; 29(20):2787-94; Ribas et al., Anti-CTLA4 Antibody Clinical Trials in Melanoma. Update Cancer Ther. 2007; 2(3):133-9; Paller et al., Hum Vaccin Immunother. 2012; 8(4):509-19). Immunotherapy relies on the cytotoxic potential of activated T cells, which scavenge to recognize and reject tumor associated or specific antigens (TAAs or TSAs). Unlike most drug agents, activated T cells can traverse the blood brain barrier (BBB) via integrin (i.e., LFA-1, VLA-4) binding of ICAMs/VCAMs (Sampson et al., Neuro Oncol. 2011; 13(3):324-33; Ransohoff et al., Nature Reviews Immunology. 2003; 3(7):569-81; Miao et al., PloS one. 2014; 9 (4):e94281). T cells can be ex vivo activated in co-culture with dendritic cells (DCs) presenting TAAs/TSAs (Mitchell et al., Nature. 2015; 519(7543):366-9) or through transduction with a chimeric antigen receptor (CAR) (Grupp et al., The New England journal of medicine. 2013; 368(16):1509-18). Alternatively, T cells can be endogenously activated using cancer vaccines; but, in a randomized phase III trial for patients with primary GBM, peptide vaccines targeting the tumor specific EGFRVIII surface antigen failed to mediate enhanced survival benefits over control vaccines (Weller et al., The Lancet Oncology. 2017; 18(10):1373-85). The EGFRVIII vaccine's failure to mediate anti-tumor efficacy highlights the challenge of therapeutic cancer vaccines.

While prophylactic cancer vaccines work to prevent malignancies (i.e., HPV vaccine to prevent cervical cancer), the vaccines require several boosts over months to years to confer protection in immune-replete patients. Furthermore, therapeutic cancer vaccines must induce immunologic response much more rapidly against malignancies (i.e., GBM) that are rapidly evolving (Sayour et al., Int J Mol Sci. 2018; 19 (10)). Moreover, GBMs are a highly invasive and heterogenous tumors associated with profound systemic/intratumoral suppression that can stymie a nascent immunotherapeutic response (Chongsathidkiet et al., Nature Medicine. 2018; 24(9):1459-68; Learn et al., Clinical cancer research: an official journal of the American Association for Cancer Research. 2006; 12(24):7306-15).

RNA vaccines have several advantages over traditional modalities. RNA has potent effects on both the innate and adaptive immune system. RNA can act as a toll-like receptor (TLR) agonist for receptors 3, 7, and 8 inducing potent TLR dependent innate immunity (24). RNA can also stimulate intracellular pathogen recognition receptors (i.e., melanoma differentiation antigen 5 (MDA-5) and retinoic acid inducible gene I (RIG-I)) and culminates in activating both helper-CD4 and cytotoxic CD8 T cell responses (Strobel et al., Gene therapy. 2000; 7(23):2028-35; Mitchell et al., The Journal of Clinical Investigation. 2000; 106(9):1065-9; Kim et al., Oncogene. 1998; 17(24):3125-35). Unlike DNA vaccines mired by having to cross both cellular and nuclear membranes, RNA only requires access to the cytoplasm and carries a significant safety advantage since it cannot be integrated into the host-genome (Sayour et al., Immunotherapy for Pediatric Brain Tumors. Brain Sci. 2017; 7 (10). Epub 2017/10/27). Unlike many peptide vaccines, which have only been developed for specific HLA haplotypes (i.e. HLA-A2), RNA bypasses MHC class restriction and can be leveraged for the population at large (Sayour et al., Immunotherapy for Pediatric Brain Tumors. Brain Sci. 2017; 7 (10). Epub 2017/10/27). One drawback to RNA is its lack of stability making it difficult to administer ‘naked’ RNA directly to patients. Since cancer vaccines must localize to antigen presenting cells (APCs) where RNA must be translated, processed and presented on MHC class I and II molecules, degradation continues to be a potent barrier for development of new mRNA technologies. To overcome these limitations, investigators within the University of Florida Brain Tumor Immunotherapy Program (UFBTIP) have developed RNA-loaded dendritic cell (DC) vaccines for the treatment of brain tumors (NCT03334305, PI: Sayour) (Sampson et al., Journal of clinical oncology: official journal of the American Society of Clinical Oncology. 2010; 28(31):4722-9; Fecci et al., Clinical cancer research: an official journal of the American Association for Cancer Research. 2007; 13(7):2158-67; Mitchell et al., Blood. 2011; 118(11):3003-12. Epub 2011/07/20; Nair et al., Clinical cancer research: an official journal of the American Association for Cancer Research. 2014. doi: 10.1158/1078-0432.CCR-13-3268. PubMed PMID: 24658154; Sanchez-Perez et al., PloS one. 2013; 8 (3):e59082; Fecci et al., Clinical cancer research: an official journal of the American Association for Cancer Research. 2006; 12 (14 Pt 1):4294-305)). It has been shown that total tumor derived mRNA (prepared autologously to represent a personalized tumor specific transcriptome) can be amplified to clinical-scale from few cells (˜500 tumor cells) providing a renewable antigen specific resource for DC vaccine production. While ex vivo generation of RNA-loaded DCs holds considerable promise, the advancement of cellular therapeutics is fraught with developmental challenges making it difficult to generate vaccines for the population at large.

To circumvent the challenges of cellular therapeutics, nanocarriers have been developed as RNA delivery vehicles but translation of nanoparticles (NPs) into human clinical trials has lagged due to unknown biologic reactivity of novel NP designs. Alternatively, simple biodegradable lipid-NPs have been developed as cationic and anionic cancer vaccine formulations. Cationic formulations have been manufactured to shield mRNA inside the lipid core while anionic formulations have been manufactured to tether mRNA to the particle surface. However, cationic formulations have been mired by poor immunogenicity, and anionic formulations remain encumbered by the profound intratumoral and systemic immunosuppression that may stymie an activated T cell response.

Thus, there is a need in the art for new RNA-NPs that overcome the aforementioned limitations.

### SUMMARY

The present disclosure provides a nanoparticle comprising a positively-charged surface and an interior comprising (i) a core and (ii) at least two nucleic acid layers, wherein each nucleic acid layer is positioned between a cationic lipid bilayer. In exemplary embodiments, the nanoparticle of the present disclosure comprises an interior comprising alternating nucleic acid layers and cationic lipid bilayers. In exemplary embodiments, the nanoparticle comprises at least three nucleic acid layers, each of which is positioned between a cationic lipid bilayer. In exemplary aspects, the nanoparticle comprises at least four or five or more nucleic acid layers, each of which is positioned between a cationic lipid bilayer. In various aspects, the outermost layer of the nanoparticle comprises a cationic lipid bilayer. In various instances, the surface comprises a plurality of hydrophilic moieties of the cationic lipid of the cationic lipid bilayer. In exemplary aspects, the core comprises a cationic lipid bilayer. In various aspects, the outermost region of the core comprises a cationic lipid bilayer. In some instances, the outermost region of the core comprise a cationic lipid bilayer comprising DOTAP. Optionally, the core comprises less than about 0.5 wt % nucleic acid. In exemplary aspects, the core comprises (i) a therapeutic agent or (ii) a diagnostic agent (e.g., an imaging agent), or (iii) a combination thereof. Suitable therapeutic agents and diagnostic agents are described herein. In exemplary aspects, the therapeutic agents comprise or are nucleic acids. Optionally, the therapeutic agents are antisense oligonucleotides (ASOs) or siRNAs. In various instances, the ASOs or siRNAs are not the same nucleic acids present in the alternating nucleic acid layers—cationic lipid bilayers. In exemplary instances, the ASOs or siRNAs are the same nucleic acids present in the alternating nucleic acid layers—cationic lipid bilayers. In various aspects, the core comprises iron oxide nanoparticles (IONPs) which are useful for imaging tissue or cells via, e.g., magnetic resonance imaging (MRI). Optionally, the IONPs are coated with a fatty acid, e.g., a C8-C30 fatty acid. In various aspects, the fatty acid is oleic acid. In various aspects, the core comprises a plurality of IONPs (optionally coated with oleic acid) wherein the plurality is held together by a lipid, e.g., a cationic lipid. Optionally, the plurality of IONPs (optionally coated with oleic acid) are held together by DOTAP. The diameter of the nanoparticle, in various aspects, is about 50 nm to about 250 nm in diameter, optionally, about 70 nm to about 200 nm in diameter. In exemplary instances, the nanoparticle is characterized by a zeta potential of about +40 mV to about +60 mV, optionally, about +45 mV to about +55 mV. The nanoparticle in various instances, has a zeta potential of about 50 mV. In some aspects, the nucleic acid molecules are present at a nucleic acid molecule:cationic lipid ratio of about 1 to about 5 to about 1 to about 20, optionally, about 1 to about 15, about 1 to about 10 or about 1 to about 7.5. In various aspects, the nucleic acid molecules are RNA molecules, optionally, messenger RNA (mRNA). In various aspects, the mRNA is in vitro transcribed mRNA wherein the in vitro transcription template is cDNA made from RNA extracted from a tumor cell. In various aspects, the nanoparticle comprises a mixture of RNA which is RNA isolated from a tumor of a human, optionally, a malignant brain tumor, optionally, a glioblastoma, medulloblastoma, diffuse intrinsic pontine glioma, or a peripheral tumor with metastatic infiltration into the central nervous system.

The present disclosure also provides a method of making a nanoparticle comprising a positively-charged surface and an interior comprising (i) a core and (ii) at least two nucleic acid layers, wherein each nucleic acid layer is positioned between a cationic lipid bilayer, said method comprising: (A) mixing nucleic acid molecules and liposomes at a RNA:liposome ratio of about 1 to about 5 to about 1 to about 20, optionally, about 1 to about 15, about 1 to about 10, or about 1 to about 7.5, to obtain a RNA-coated liposomes, wherein the liposomes are made by a process of making liposomes comprising drying a lipid mixture comprising a cationic lipid and an organic solvent by evaporating the organic solvent under a vacuum; and (B) mixing the RNA-coated liposomes with a surplus amount of liposomes. In exemplary aspects, the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent. In various instances, the process of making liposomes further comprises rehydrating the lipid mixture with a rehydration solution to form a rehydrated lipid mixture and then agitating, resting, and sizing the rehydrated lipid mixture. Optionally, sizing the rehydrated lipid mixture comprises sonicating, extruding and/or filtering the rehydrated lipid mixture.

Further provided herein are nanoparticles made by the presently disclosed method of making a nanoparticle. Additionally provided herein is a cell comprising a nanoparticle of the present disclosure. Optionally, the cell is an antigen presenting cell (APC), e.g., a dendritic cell (DC). The present disclosure also provides a population of cells, wherein at least 50% of the population are cells according to the present disclosure.

The present disclosure provides a pharmaceutical composition comprising a plurality of nanoparticles according to the present disclosure and a pharmaceutically acceptable carrier, diluent, or excipient. In various aspects, the composition comprises about 10¹⁰ nanoparticles per mL to about 10¹⁵ nanoparticles per mL, optionally about 10¹¹ nanoparticles ±10% per mL.

A method of increasing an immune response, such as an immune response against a tumor, in a subject is provided by the present disclosure. In exemplary embodiments, the method comprises administering to the subject the pharmaceutical composition of the present disclosure. In exemplary aspects, the nucleic acid molecules are mRNA. Optionally, the composition is systemically administered to the subject. For example, the composition is administered intravenously. In various aspects, the pharmaceutical composition is administered in an amount which is effective to activate dendritic cells (DCs) in the subject. In various instances, the immune response is a T cell-mediated immune response. Optionally, the T cell-mediated immune response comprises activity by tumor infiltrating lymphocytes (TILs).

The present disclosure also provides a method of delivering RNA molecules to an intra-tumoral microenvironment, lymph node, and/or a reticuloendothelial organ. In exemplary embodiments, the method comprises administering to the subject a presently disclosed pharmaceutical composition. Optionally, the reticuloendothelial organ is a spleen or liver.

A method of treating a subject with a disease is furthermore provided herein. In exemplary embodiments, the method comprises delivering RNA molecules to cells of the subject according to the presently disclosed method of delivering RNA molecules to an intra-tumoral microenvironment, lymph node, and/or a reticuloendothelial organ. In various aspects, RNA molecules are ex vivo delivered to the cells and the cells are administered to the subject. In exemplary embodiments, the method comprises administering to the subject a pharmaceutical composition of the present disclosure in an amount effective to treat the disease in the subject. In various instances, the subject has a cancer or a tumor, optionally, a malignant brain tumor, optionally, a glioblastoma, medulloblastoma, diffuse intrinsic pontine glioma, or a peripheral tumor with metastatic infiltration into the central nervous system.

Additional embodiments and aspects of the presently disclosed pharmaceutical compositions and methods are provided below.

### BRIEF DESCRIPTION OF THE DRAWINGS

FIG. 1A is a series of illustrations of a lipid bilayer, liposome and a general scheme leading to multilamellar (ML) RNA NPs (boxed).

FIG. 1B is a pair of CEM images of uncomplexed NPs (left) and ML RNA NPs (right).

FIG. 2A is an illustration of a general scheme leading to cationic RNA lipoplexes.

FIG. 2B is an illustration of a general scheme leading to cationic RNA lipoplexes.

FIGS. 2C-2D are CEM images. FIG. 2C is a CEM image of uncomplexed NPs, FIG. 2D is a CEM image of RNA LPXs, and FIG. 2E is a CEM image of ML RNA NPs.

FIG. 2F is a graph of the % CD86+ of CD11c+MHC Class II+ splenocytes present in the spleens of mice treated with ML RNA NPs (ML RNA-NPs), RNA LPXs, anionic LPXs, or of untreated mice.

FIG. 2G is a graph of the % CD44+CD62L+ of CD8+ splenocytes present in the spleens of mice treated with ML RNA NPs (ML RNA-NPs), RNA LPXs, anionic LPXs, or of untreated mice.

FIG. 2H is a graph of the % CD44+CD62L of CD4+ splenocytes present in the spleens of mice treated with ML RNA NPs (ML RNA-NPs), RNA LPXs, anionic LPXs, or of untreated mice.

FIG. 2I is a graph of the % survival of mice treated with ML RNA NPs (ML RNA-NPs), RNA LPXs, anionic LPXs, or of untreated mice.

FIG. 2J is a graph of the amount of IFN-α produced in mice upon treatment with ML RNA NPs (ML RNA-NPs), RNA LPXs, anionic LPXs, or of untreated mice.

FIG. 3A is a pair of photographs of lungs of mice treated with ML RNA NPs or of untreated mice.

FIG. 3B is a graph of the % central memory T cells (CD62L+CD44+ of CD3+ cells) present in mice treated with ML RNA NPs loaded with tumor specific RNA or with ML RNA NPs with non-specific RNA (GFP RNA) or of untreated mice.

FIG. 3C is a graph of the % survival of mice treated with ML RNA NPs loaded with tumor specific RNA or with ML RNA NPs with non-specific RNA (GFP RNA) or of untreated mice.

FIG. 3D is a graph of the % survival of mice treated with ML RNA NPs loaded with tumor specific RNA or with ML RNA NPs with non-specific RNA (GFP RNA) or of untreated mice. This model is different from the one used to obtain the data of FIG. 3C.

FIGS. 4A-4D are graphs. FIG. 4A is a graph of the % expression of CD8 or CD44 and CD8 of CD3+ cells plotted as a function of time post administration of ML RNA NPs. FIG. 4B is a graph of the % expression of PDL1, MHC II, CD86 or CD80 of CD11c+ cells plotted as a function of time post administration of ML RNA NPs. FIG. 4C is a graph of the % expression of CD44 and CD8 of CD3+ cells plotted as a function of time post administration of ML RNA NPs. FIG. 4D is a graph of the % survival of a canine treated with ML RNA NPs compared to the median survival (dotted line).

FIG. 5 is a CEM image of ML RNA NPs and point to examples with several layers.

FIG. 6 is a cartoon delineating the generation of personalized tumor mRNA loaded NPs. From as few as 100-500 biopsied brain tumor cells, total RNA is extracted and a cDNA library is generated from which copious amounts of mRNA (representing a personalized tumor specific transcriptome) can be amplified. Negatively charged tumor mRNA is then encapsulated into positively charged lipid NPs. NPs encapsulate RNA through electrostatic interaction and are administered intravenously (iv) for uptake by dendritic cells (DCs) in reticuloendothelial organs (i.e., liver spleen and lymph nodes). The RNA is then translated and processed by a DC's intracellular machinery for presentation of peptides onto MHC Class I and II molecules, which activate CD4 and CD8+ T cells.

FIG. 7A is a timeline of the long-term survivor treatment. First and Second tumor inoculations are shown. FIG. 7B is a graph of the percent survival of animals after the second tumor inoculation for each of the three groups of mice: two groups treated before 2^{nd} tumor inoculation with ML RNA NPs comprising non-specific RNA (RNA not specific to the tumor in the subject; Green Fluorescence Protein (GFP) or pp65) and one group treated before 2^{nd} tumor inoculation with ML RNA NPs comprising tumor specific RNA or untreated animals prior to 2^{nd} tumor inoculation. Control group survival percentage is noted as “Untreated”.

FIG. 8 is a series of images depicting the localization of anionic LPX in mice upon administration.

FIG. 9 is an image of iron oxide nanoparticles held together by a lipid coating of DOTAP.

FIG. 10 demonstrates multi-lamellar RNA NPs form complex structures that coil mRNA into multi-lamellar vesicles enhancing payload delivery. The bar graph illustrates gene expression (luminescence) for anionic RNA-LPS (first bar on left), RNA-lipoplex (second bar), RNA-NPs (lo) (third bar), and RNA-NPs (high) (fourth bar).

FIG. 11 demonstrates multi-lamellar RNA NPs mediate increased DC activation and IFN-α release. RNA/anionic lipoplex (LPX) or RNA-NPs were i.v. (intravenously) administrated once weekly (×3) to C57Bl/6 mice, and spleens were harvested one week later for assessment of activated DCs (left). Serum was drawn 6 h after the initial treatment for IFN-α assessment by ELISA (right).

FIG. 12 demonstrates multi-lamellar RNA-NPs are superior to LPX and peptide based vaccines in eliciting antigen specific T cells. RNA/anionic lipoplex (LPX) (left) or peptide based vaccines (right) formulated in complete Freund's adjuvant (CFA) were compared with OVA specific RNA-NPs. Animals (n=5-8/group) received 10⁷OT-Is before assessment of tetramer positive (OVA specific) T cells one week after last vaccine.

FIG. 13 demonstrates RNA-NPs induce memory re-stimulation response against CMV matrix protein pp65. Weekly pp65 RNA-NPs (×3) were administered to naïve C57/BI/6 mice, and splenocytes were harvested one week later for culture with overlapping pp65 peptide pool and assessment of IFN-γ (*p<0.05, **p<0.01, Mann Whitney).

FIG. 14 demonstrates multi-lamellar tumor specific mRNA-NPs mediate superior efficacy. Different lipoplexes (LPX) or RNA-NPs were loaded with tumor specific mRNA and compared in a therapeutic lung cancer model (K7M2) (n=8/group). Each vaccine was iv administered weekly (×3), **p<0.01, Gehan-Wilcoxon test.

FIG. 15A-15C demonstrate charge modified RNA-NPs can be directed to, e.g., the lung or the spleen. RNA-NPs were injected iv into C57Bl/6 mice (n=3-4/group). Reticuloendothelial organs (lymph nodes, spleens, and livers) were harvested within 24 h for assessment of CD11c cells expressing activation marker CD86 (*p<0.05, **p<0.01, Mann-Whitney test) from lymph nodes (FIG. 15A), splenocytes (FIG. 15B), or liver cells (FIG. 15C). The data establish that the constructs of the disclosure can delivered to reticuloendothelial organs with only a single administration.

FIG. 16 illustrates that full-length LAMP conjugated pp65 appears to induce greater percentage of antigen specific T cells. Full length LAMP conjugated RNA for pp65 was i.v. administered to naïve mice (n=5/group) once weekly (×3) and spleens were harvested for re-stimulation with overlapping pp65 peptide pool (*p<0.05, Mann-Whitney test). The graph compares IFN production in subjects administered NP alone, RNA-NP, or LAMP RNA-NP.

FIGS. 17A and 17B are graphs illustrating % OVA specific Tetramer+CD8 cells in subjects administered NP alone and RNA-NP in MDAS knock-out subjects. FIG. 17A-T cells alone; FIG. 17B—following restimulation assay with B16F10-OVA.

FIG. 18: RNA-NPs mediate efficacy independent of TLR7. Wild-type TLR7+/+C57Bl/6 mice (n=8/group) were s.c implanted with tumors derived from B16F10 and tumor volumes were compared with TLR7−/− knock out (KO) mice (n=5-8/group) on C57Bl/6 background; both groups of animals were i.v. treated with RNA-NPs weekly (×3) versus KO mice receiving NP alone (***p<0.001, two-way ANOVA).

FIGS. 19A and 19B: RNA-NPs mediate IFNAR1 dependent response independent of TLR7. (FIG. 19A) K7M2 (1.25×10⁶ cells) were inoculated into the lungs of Balb/c (n=7/group) mice via tail vein injection, and i.v. treated with weekly RNA-NPs (×3) with or without biweekly IFN-α blocking antibodies (IFNAR1 mAbs), ***pWild-type TLR7+/+C57Bl/6 mice (n=8) were implanted with B16F0 melanomas and survival outcomes were compared with TLR7−/− knock out (KO) mice (n=5/group) on C57Bl/6 background, both groups of animals were i.v. treated with RNA-NPs weekly (×3) versus KO mice receiving NP alone (*p).

FIGS. 20A and 20B: RNA-NPs mediate memory recall response. (FIG. 20A) Balb/c mice (5-8/group) inoculated with K7M2 lung tumors were subsequently i.v. vaccinated with three weekly RNA-NPs and spleens were harvested one week after the 3rd vaccine for analysis of ex vivo memory recall response to tumor antigens (K7M2) versus control tumor (B16F0) by IFN-γ (*p<0.05, Mann Whitney test). (FIG. 20B) Long-term surviving animals previously treated with RNA-NPs (n=7), were re-challenged with i.v. administration of K7M2 tumor cells (1.25×10⁶ cells), and compared with a new cohort of untreated mice (n=8) inoculated with K7M2 tumors (****p<0.001, Gehan-Breslow-Wilcoxon test).

### DETAILED DESCRIPTION

The present disclosure relates to nanoparticles comprising a cationic lipid and nucleic acids. As used herein the term “nanoparticle” refers to a particle that is less than about 1000 nm in diameter. As the nanoparticles of the present disclosure comprise cationic lipids that have been processed to induce liposome formation, the presently disclosed nanoparticles in various aspects comprise liposomes. Liposomes are artificially-prepared vesicles which, in exemplary aspects, are primarily composed of a lipid bilayer. Liposomes in various instances are used as a delivery vehicle for the administration of nutrients and pharmaceutical agents. In various aspects the liposomes of the present disclosure are of different sizes and the composition may comprise one or more of (a) a multilamellar vesicle (MLV) which may be hundreds of nanometers in diameter and may contain a series of concentric bilayers separated by narrow aqueous compartments, (b) a small unicellular vesicle (SUV) which may be smaller than 50 nm in diameter, and (c) a large unilamellar vesicle (LUV) which may be between 50 and 500 nm in diameter. Liposomes in various instances are designed to comprise opsonins or ligands in order to improve the attachment of liposomes to unhealthy tissue or to activate events such as, but not limited to, endocytosis. In exemplary aspects, liposomes contain a low or a high pH in order to improve the delivery of the pharmaceutical formulations. In various instances, liposomes are formulated depending on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In exemplary embodiments, the nanoparticle comprises a surface and an interior comprising (i) a core and (ii) at least two nucleic acid layers, optionally, more than two nucleic acid layers. In exemplary instances, each nucleic acid layer is positioned between a lipid layer, e.g., a cationic lipid layer. In exemplary aspects, the nanoparticles are multilamellar comprising alternating layers of nucleic acid and lipid. In exemplary embodiments, the nanoparticle of the present disclosure comprises an interior comprising alternating nucleic acid layers and cationic lipid bilayers. In exemplary embodiments, the nanoparticle comprises at least three nucleic acid layers, each of which is positioned between a cationic lipid bilayer. In exemplary aspects, the nanoparticle comprises at least four or five nucleic acid layers, each of which is positioned between a cationic lipid bilayer. In exemplary aspects, the nanoparticle comprises at least more than five (e.g., 6, 7, 8, 9, 10, 11, 12, 13, 14, 15, 16, 17, 18, 19, 20, or more) nucleic acid layers, each of which is positioned between a cationic lipid bilayer. As used herein the term “cationic lipid bilayer” is meant a lipid bilayer comprising, consisting essentially of, or consisting of a cationic lipid or a mixture thereof. Suitable cationic lipids are described herein. As used herein the term “nucleic acid layer” is meant a layer of the presently disclosed nanoparticle comprising, consisting essentially of, or consisting of a nucleic acid, e.g., RNA.

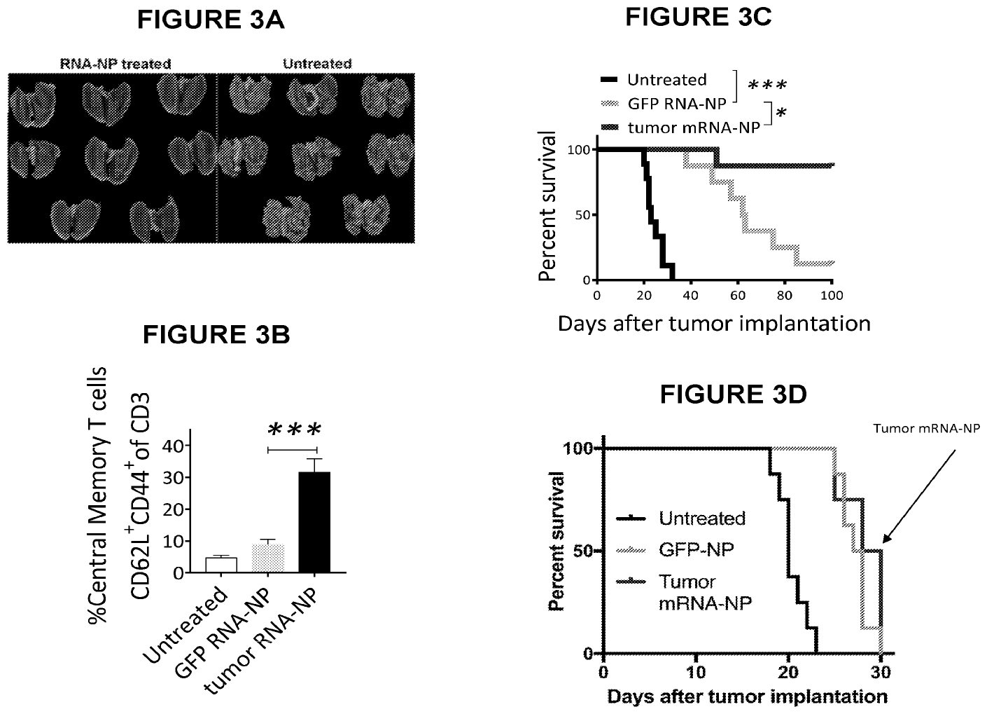

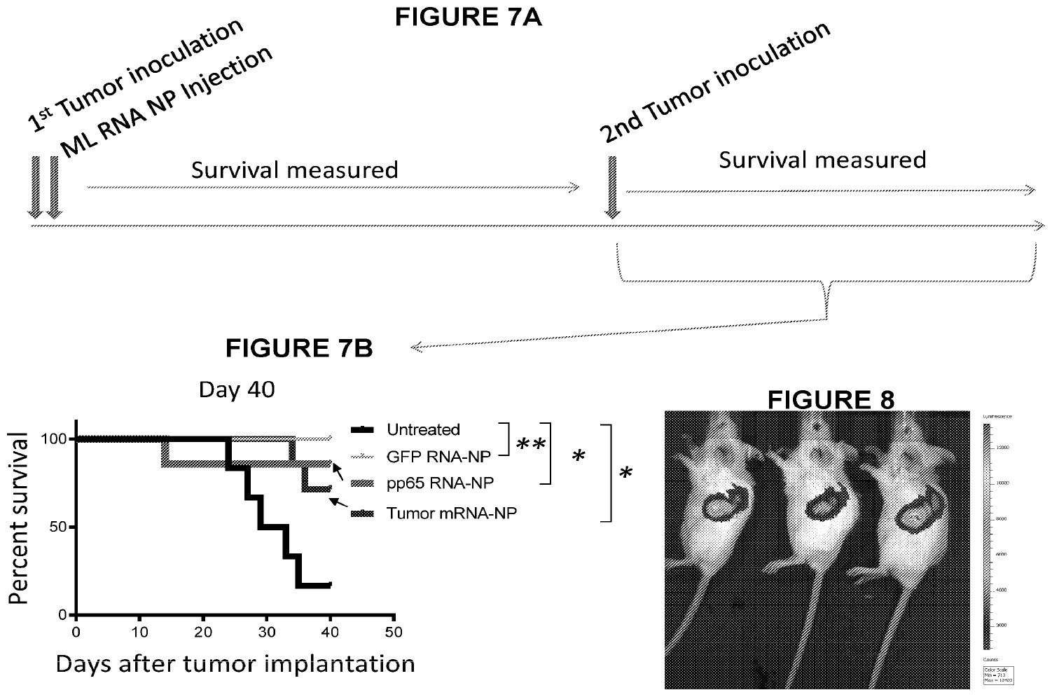

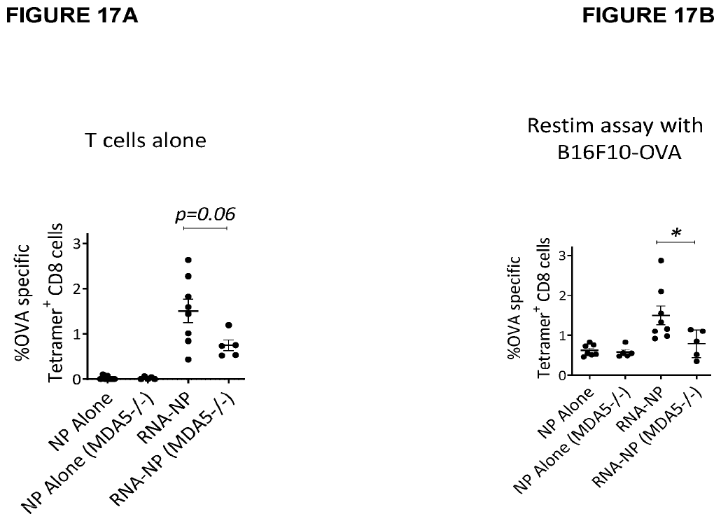

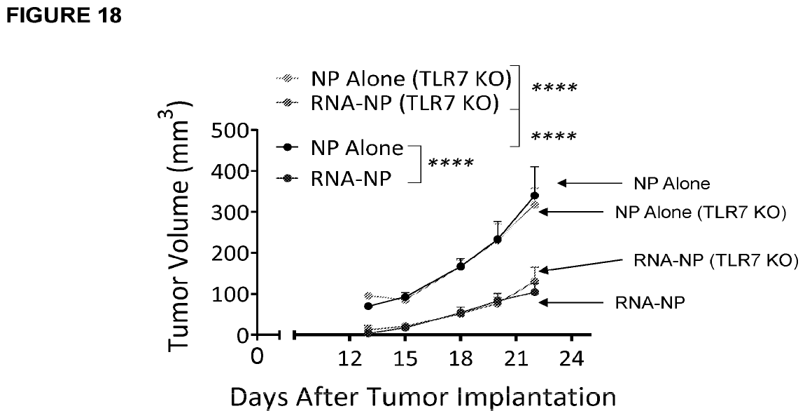

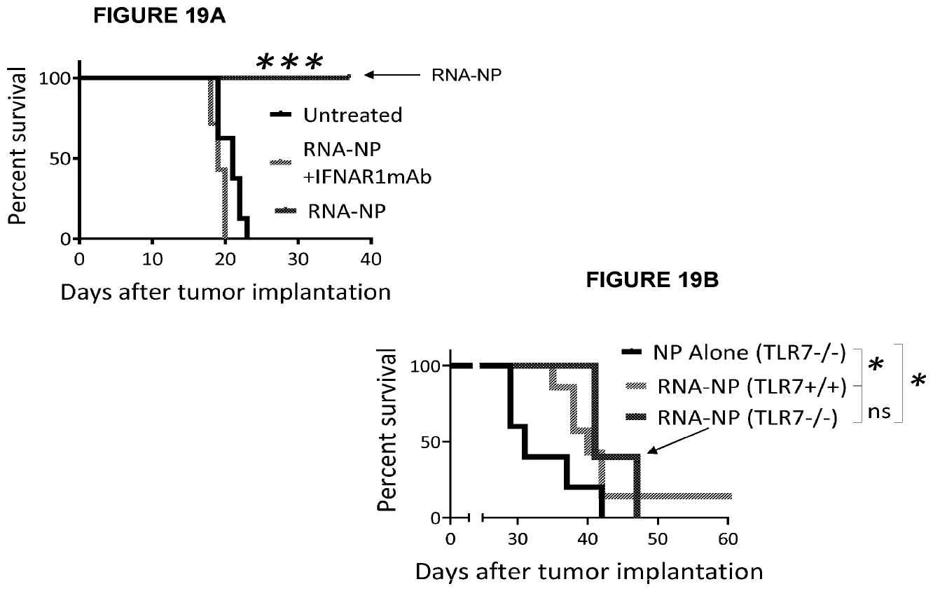

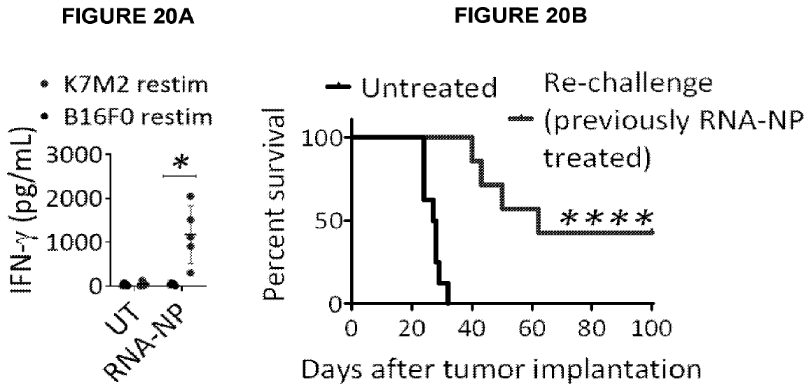

The unique structure of the nanoparticle of the present disclosure results in mechanistic differences in how the multi-lamellar nanoparticles exert a biological effect. Previously described RNA-based nanoparticles exert their effect, at least in part, through the toll-like receptor 7 (TLR7) pathway. Surprisingly, the multi-lamellar nanoparticles of the instant disclosure mediate efficacy independent of TLR7. See, e.g., FIGS. 18 and 19A-19B. While not wishing to be bound to any particular theory, intracellular pathogen recognition receptors (PRRs), such as MDA-5, appear more relevant to biological activity of the multi-lamellar nanoparticles than TLRs. See, e.g., FIG. 17. This likely allows ML RNA-NPs to stimulate multiple intracellular PRRs (i.e., RIG-1, MDA-5) as opposed to singular TLRs (i.e., TLR7 in the endosome) culminating in greater release of type I interferons and induction of more potent innate immunity (FIG. 11). This allows irrelevant species of mRNA to elicit anti-tumor activity (FIGS. 3B, 3C, and 7B) and tumor specific RNA-NPs to demonstrate superior efficacy with long-term survivor benefit (FIG. 14).

In various aspects, the presently disclosed nanoparticle comprises a positively-charged surface. In some instances, the positively-charged surface comprises a lipid layer, e.g., a cationic lipid layer. In various aspects, the outermost layer of the nanoparticle comprises a cationic lipid bilayer. Optionally, the cationic lipid bilayer comprises DOTAP. In various instances, the surface comprises a plurality of hydrophilic moieties of the cationic lipid of the cationic lipid bilayer. In some aspects, the core comprises a cationic lipid bilayer. In various aspects, the outermost region of the core comprises a cationic lipid bilayer. In some instances, the outermost region of the core comprise a cationic lipid bilayer comprising DOTAP. In various instances, the core lacks nucleic acids. Optionally, the core comprises less than about 0.5 wt % nucleic acid. In exemplary aspects, the core comprises (i) a therapeutic agent or (ii) a diagnostic agent (e.g., an imaging agent) or (iii) a combination thereof. Suitable therapeutic agents and diagnostic agents are described herein. In exemplary aspects, the therapeutic agents comprise or are nucleic acids. Optionally, the therapeutic agents are antisense oligonucleotides (ASOs) or siRNAs. In various instances, the ASOs or siRNAs are not the same nucleic acids present in the alternating nucleic acid layers—cationic lipid bilayers. In exemplary instances, the ASOs or siRNAs are the same nucleic acids present in the alternating nucleic acid layers—cationic lipid bilayers. In various aspects, the core comprises iron oxide nanoparticles (IONPs) which are useful for imaging tissue or cells via, e.g., magnetic resonance imaging (MRI). Optionally, the IONPs are coated with a fatty acid, e.g., a C8-C30 fatty acid. In various aspects, the fatty acid is oleic acid. In various aspects, the core comprises a plurality of IONPs (optionally coated with oleic acid) wherein the plurality is held together by a lipid, e.g., a cationic lipid. Optionally, the plurality of IONPs (optionally coated with oleic acid) are held together by DOTAP. Further description of cores comprising therapeutic agents and diagnostic agents are provided below.

In exemplary aspects, the nanoparticle has a diameter within the nanometer range and accordingly in certain instances are referred to herein as “nanoliposomes” or “liposomes”. In exemplary aspects, the nanoparticle has a diameter between about 50 nm to about 500 nm, e.g., about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 500 nm, about 150 nm to about 500 nm, about 200 nm to about 500 nm, about 250 nm to about 500 nm, about 300 nm to about 500 nm, about 350 nm to about 500 nm, about 400 nm to about 500 nm. In exemplary aspects, the nanoparticle has a diameter between about 50 nm to about 300 nm, e.g., about 100 nm to about 250 nm, about 110 nm±5 nm, about 115 nm±5 nm, about 120 nm±5 nm, about 125 nm±5 nm, about 130 nm±5 nm, about 135 nm 5 nm, about 140 nm 5 nm, about 145 nm±5 nm, about 150 nm±5 nm, about 155 nm±5 nm, about 160 nm±5 nm, about 165 nm±5 nm, about 170 nm±5 nm, about 175 nm±5 nm, about 180 nm±5 nm, about 190 nm±5 nm, about 200 nm±5 nm, about 210 nm±5 nm, about 220 nm±5 nm, about 230 nm±5 nm, about 240 nm±5 nm, about 250 nm±5 nm, about 260 nm±5 nm, about 270 nm±5 nm, about 280 nm±5 nm, about 290 nm±5 nm, about 300 nm±5 nm. In exemplary aspects, the nanoparticle is about 50 nm to about 250 nm in diameter. In some aspects, the nanoparticle is about 70 nm to about 200 nm in diameter.

In exemplary aspects, the nanoparticle is present in a pharmaceutical composition comprising a heterogeneous mixture of nanoparticles ranging in diameter, e.g., about 50 nm to about 500 nm or about 50 nm to about 250 nm in diameter. Optionally, the pharmaceutical composition comprises a heterogeneous mixture of nanoparticles ranging from about 70 nm to about 200 nm in diameter.

In exemplary instances, the nanoparticle is characterized by a zeta potential of about +40 mV to about +60 mV, e.g., about +40 mV to about +55 mV, about +40 mV to about +50 mV, about +40 mV to about +50 mV, about +40 mV to about +45 mV, about +45 mV to about +60 mV, about +50 mV to about +60 mV, about +55 mV to about +60 mV. In exemplary aspects, the nanoparticle has a zeta potential of about +45 mV to about +55 mV. The nanoparticle in various instances, has a zeta potential of about +50 mV. In various aspects, the zeta potential is greater than +30 mV or +35 mV. The zeta potential is one parameter which distinguishes the nanoparticles of the present disclosure and those described in Sayour et al., Oncoimmunology 6(1): e1256527 (2016).

In exemplary embodiments, the nanoparticles comprise a cationic lipid. In some embodiments, the cationic lipid is a low molecular weight cationic lipid such as those described in U.S. Patent Application No. 20130090372, the contents of which are herein incorporated by reference in their entirety. The cationic lipid in exemplary instances is a cationic fatty acid, a cationic glycerolipid, a cationic glycerophospholipid, a cationic sphingolipid, a cationic sterol lipid, a cationic prenol lipid, a cationic saccharolipid, or a cationic polyketide. In exemplary aspects, the cationic lipid comprises two fatty acyl chains, each chain of which is independently saturated or unsaturated. In some instances, the cationic lipid is a diglyceride. For example, in some instances, the cationic lipid may be a cationic lipid of Formula I or Formula II:

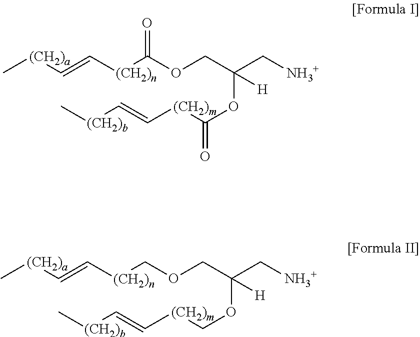

wherein each of a, b, n, and m is independently an integer between 2 and 12 (e.g., between 3 and 10). In some aspects, the cationic lipid is a cationic lipid of Formula I wherein each of a, b, n, and m is independently an integer selected from 3, 4, 5, 6, 7, 8, 9, and 10. In exemplary instances, the cationic lipid is DOTAP (1,2-dioleoyl-3-trimethylammonium-propane), or a derivative thereof. In exemplary instances, the cationic lipid is DOTMA (1,2-di-O-octadecenyl-3-trimethylammonium propane), or a derivative thereof.

In some embodiments, the nanoparticles comprise liposomes formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety). In some embodiments, the nanoparticles comprise liposomes formed from the synthesis of stabilized plasmid-lipid particles (SPLP) or stabilized nucleic acid lipid particle (SNALP) that have been previously described and shown to be suitable for oligonucleotide delivery in vitro and in vivo. The nanoparticles in some aspects are composed of 3 to 4 lipid components in addition to the nucleic acid molecules. In exemplary aspects, the liposome comprises 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. In exemplary instances, the liposome comprises 48% cholesterol, 20% DSPC, 2% PEG-c-DMA, and 30% cationic lipid, where the cationic lipid can be 1,2-distearloxy-N,N-dimethylaminopropane (DSODMA), DODMA, DLin-DMA, or 1,2-dilinolenyloxy-3-dimethylaminopropane (DLenDMA), as described by Heyes et al.

In some embodiments, the liposomes comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some embodiments, the liposomes may comprise a percentage of cholesterol selected from the group consisting of 28.5%, 31.5%, 33.5%, 36.5%, 37.0%, 38.5%, 39.0% and 43.5%. In some embodiments, the liposomes may comprise from about 5.0% to about 10.0% DSPC and/or from about 7.0% to about 15.0% DSPC.

In some embodiments, the liposomes are DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5 (12)1708-1713); herein incorporated by reference in its entirety) and hyaluronan-coated liposomes (Quiet Therapeutics, Israel).

In various instances, the cationic lipid comprises 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

The liposome in various aspects comprises DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some aspects, the liposome comprises a cationic lipid such as, but not limited to, DLin-DMA, DLin-D-DMA, DLin-MC3-DMA, DLin-KC2-DMA, DODMA and amino alcohol lipids. The amino alcohol cationic lipid comprises in some aspects lipids described in and/or made by the methods described in U.S. patent publication No. U.S. 20130150625, herein incorporated by reference in its entirety. As a non-limiting example, the cationic lipid in certain aspects is 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in U.S. 20130150625); 2-amino-3-[(9Z)-octadec-9-en-1-yloxy]-2-{[(9Z)-octadec-9-en-1-yloxy]methyl}propan-1-ol (Compound 2 in U.S. 20130150625); 2-amino-3-[(9Z,12Z)-octadeca-9,12-dien-1-yloxy]-2-[(octyloxy)methyl]propan-1-ol (Compound 3 in US 20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in U.S. 20130150625); or any pharmaceutically acceptable salt or stereoisomer thereof.

In various embodiments, the liposome comprises (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid: 5-25% neutral lipid: 25-55% sterol; 0.5-15% PEG-lipid.

In some embodiments, the liposome comprises from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In some embodiments, the liposome comprises from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In some embodiments, the formulation includes from about 5% to about 50% on a molar basis of the sterol (e.g., about 15 to about 45%, about 20 to about 40%, about 40%, about 38.5%, about 35%, or about 31% on a molar basis). An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis). In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety).

In exemplary aspects, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N,N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-methyloctyl)oxyl-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-octylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In some embodiments, the nanoparticle comprises a lipid-polycation complex. The formation of the lipid-polycation complex may be accomplished by methods known in the art and/or as described in U.S. Patent Publication No. 20120178702, herein incorporated by reference in its entirety. As a non-limiting example, the polycation may include a cationic peptide or a polypeptide such as, but not limited to, polylysine, polyornithine and/or polyarginine. In some embodiments, the composition may comprise a lipid-polycation complex, which may further include a non-cationic lipid such as, but not limited to, cholesterol or dioleoyl phosphatidylethanolamine (DOPE).

In some aspects, the nucleic acid molecules are present at a nucleic acid molecule: cationic lipid ratio of about 1 to about 5 to about 1 to about 20, optionally, about 1 to about 15, about 1 to about 10, or about 1 to about 7.5. As used herein, the term “nucleic acid molecule: cationic lipid ratio” is meant a mass ratio, where the mass of the nucleic acid molecule is relative to the mass of the cationic lipid. Also, in exemplary aspects, the term “nucleic acid molecule:cationic lipid ratio” is meant the ratio of the mass of the nucleic acid molecule, e.g., RNA, added to the liposomes comprising cationic lipids during the process of manufacturing the ML RNA NPs of the present disclosure. In exemplary aspects, the nanoparticle comprises less than or about 10 μg RNA molecules per 150 μg lipid mixture. In exemplary aspects, the nanoparticle is made by incubating about 10 μg RNA with about 150 μg liposomes. In alternative aspects, the nanoparticle comprises more RNA molecules per mass of lipid mixture. For example, the nanoparticle may comprise more than 10 μg RNA molecules per 150 μg liposomes. The nanoparticle in some instances comprises more than 15 μg RNA molecules per 150 μg liposomes or lipid mixture.

In various aspects, the nucleic acid molecules are RNA molecules, e.g., transfer RNA (tRNA), ribosomal RNA (rRNA), or messenger RNA (mRNA). In various aspects, the RNA molecules comprise tRNA, rRNA, mRNA, or a combination thereof. In various aspects, the RNA is total RNA isolated from a cell. In exemplary aspects, the RNA is total RNA isolated from a diseased cell, such as, for example, a tumor cell or a cancer cell. Methods of obtaining total tumor RNA is known in the art and described herein at Example 1.

In exemplary instances, the RNA molecules are mRNA. In various aspects, mRNA is in vitro transcribed mRNA. In various instances, the mRNA molecules are produced by in vitro transcription (IVT). Suitable techniques of carrying out IVT are known in the art. In exemplary aspects, an IVT kit is employed. In exemplary aspects, the kit comprises one or more IVT reaction reagents. As used herein, the term “in vitro transcription (IVT) reaction reagent” refers to any molecule, compound, factor, or salt, which functions in an IVT reaction. For example, the kit may comprise prokaryotic phage RNA polymerase and promoter (T7, T3, or SP6) with eukaryotic or prokaryotic extracts to synthesize proteins from exogenous DNA templates. In exemplary aspects, the RNA is in vitro transcribed mRNA, wherein the in vitro transcription template is cDNA made from RNA extracted from a tumor cell. In various aspects, the nanoparticle comprises a mixture of RNA which is RNA isolated from a tumor of a human, optionally, a malignant brain tumor, optionally, a glioblastoma, medulloblastoma, diffuse intrinsic pontine glioma, or a peripheral tumor with metastatic infiltration into the central nervous system. In various aspects, the RNA comprises a sequence encoding a poly(A) tail so that the in vitro transcribed RNA molecule comprises a poly(A) tail at the 3′ end. In various aspects, the method of making a nanoparticle comprises additional processing steps, such as, for example, capping the in vitro transcribed RNA molecules.

The mRNAs in exemplary aspects encode a protein. Optionally, the protein is selected from the group consisting of a tumor antigen, a cytokine, and a co-stimulatory molecule. In some aspects, the RNA molecule encodes a protein. The protein is, in some aspects, selected from the group consisting of a tumor antigen, a co-stimulatory molecule, a cytokine, a growth factor, a lymphokine (including, e.g., cytokines and growth factors that are effective in inhibiting tumor metastasis, or cytokines or growth factors that have been shown to have an antiproliferative effect on at least one cell population). Such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof. In exemplary aspects, the tumor antigen is an antigen derived from a viral protein, an antigen derived from point mutations, or an antigen encoded by a cancer-germline gene. In exemplary aspects, the tumor antigen is pp65, p53, KRAS, NRAS, MAGEA, MAGEB, MAGEC, BAGE, GAGE, LAGE/NY-ESO1, SSX, tyrosinase, gp100/pmel17, Melan-A/MART-1, gp75/TRP1, TRP2, CEA, RAGE-1, HER2/NEU, WT1. In exemplary aspects, the co-stimulatory molecule is selected from the group consisting of: CD80 and CD86. In some aspects, the protein is not expressed by a tumor cell or by a human. In exemplary instances, the protein is not related to a tumor antigen or cancer antigen. In some aspects, the protein is non-specific relative to a tumor or cancer. For example, the non-specific protein may be green fluorescence protein (GFP) or ovalbumin (OVA).

In various instances, the RNA molecules are antisense molecules, optionally siRNA, shRNA, miRNA, or any combination thereof. The antisense molecule can be one which mediates RNA interference (RNAi). As known by one of ordinary skill in the art, RNAi is a ubiquitous mechanism of gene regulation in plants and animals in which target mRNAs are degraded in a sequence-specific manner (Sharp, Genes Dev., 15, 485-490 (2001); Hutvagner et al., Curr. Opin. Genet. Dev., 12, 225-232 (2002); Fire et al., Nature, 391, 806-811 (1998); Zamore et al., Cell, 101, 25-33 (2000)). The natural RNA degradation process is initiated by the dsRNA-specific endonuclease Dicer, which promotes cleavage of long dsRNA precursors into double-stranded fragments between 21 and 25 nucleotides long, termed small interfering RNA (siRNA; also known as short interfering RNA) (Zamore, et al., Cell. 101, 25-33 (2000); Elbashir et al., Genes Dev., 15, 188-200 (2001); Hammond et al., Nature, 404, 293-296 (2000); Bernstein et al., Nature, 409, 363-366 (2001)). siRNAs are incorporated into a large protein complex that recognizes and cleaves target mRNAs (Nykanen et al., Cell, 107, 309-321 (2001). It has been reported that introduction of dsRNA into mammalian cells does not result in efficient Dicer-mediated generation of siRNA and therefore does not induce RNAi (Caplen et al., Gene 252, 95-105 (2000); Ui-Tei et al., FEBS Lett, 479, 79-82 (2000)). The requirement for Dicer in maturation of siRNAs in cells can be bypassed by introducing synthetic 21-nucleotide siRNA duplexes, which inhibit expression of transfected and endogenous genes in a variety of mammalian cells (Elbashir et al., Nature, 411: 494-498 (2001)).

In this regard, the RNA molecule in some aspects mediates RNAi and in some aspects is a siRNA molecule specific for inhibiting the expression of a protein. The term “siRNA” as used herein refers to an RNA (or RNA analog) comprising from about 10 to about 50 nucleotides (or nucleotide analogs) which is capable of directing or mediating RNAi. In exemplary embodiments, an siRNA molecule comprises about 15 to about 30 nucleotides (or nucleotide analogs) or about 20 to about 25 nucleotides (or nucleotide analogs), e.g., 21-23 nucleotides (or nucleotide analogs). The siRNA can be double or single stranded, preferably double-stranded.

In alternative aspects, the RNA molecule is alternatively a short hairpin RNA (shRNA) molecule specific for inhibiting the expression of a protein. The term “shRNA” as used herein refers to a molecule of about 20 or more base pairs in which a single-stranded RNA partially contains a palindromic base sequence and forms a double-strand structure therein (i.e., a hairpin structure). An shRNA can be an siRNA (or siRNA analog) which is folded into a hairpin structure. shRNAs typically comprise about 45 to about 60 nucleotides, including the approximately 21 nucleotide antisense and sense portions of the hairpin, optional overhangs on the non-loop side of about 2 to about 6 nucleotides long, and the loop portion that can be, e.g., about 3 to 10 nucleotides long. The shRNA can be chemically synthesized. Alternatively, the shRNA can be produced by linking sense and antisense strands of a DNA sequence in reverse directions and synthesizing RNA in vitro with T7 RNA polymerase using the DNA as a template.

Though not wishing to be bound by any theory or mechanism it is believed that after shRNA is introduced into a cell, the shRNA is degraded into a length of about 20 bases or more (e.g., representatively 21, 22, 23 bases), and causes RNAi, leading to an inhibitory effect. Thus, shRNA elicits RNAi and therefore can be used as an effective component of the disclosure. shRNA may preferably have a 3-protruding end. The length of the double-stranded portion is not particularly limited, but is preferably about 10 or more nucleotides, and more preferably about 20 or more nucleotides. Here, the 3-protruding end may be preferably DNA, more preferably DNA of at least 2 nucleotides in length, and even more preferably DNA of 2-4 nucleotides in length.

In exemplary aspects, the antisense molecule is a microRNA (miRNA). As used herein the term “microRNA” refers to a small (e.g., 15-22 nucleotides), non-coding RNA molecule which base pairs with mRNA molecules to silence gene expression via translational repression or target degradation. microRNA and the therapeutic potential thereof are described in the art. See, e.g., Mulligan, MicroRNA: Expression, Detection, and Therapeutic Strategies, Nova Science Publishers, Inc., Hauppauge, N.Y., 2011; Bader and Lammers, “The Therapeutic Potential of microRNAs” Innovations in Pharmaceutical Technology, pages 52-55 (March 2011).

In certain instances, the RNA molecule is an antisense molecule, optionally, an siRNA, shRNA, or miRNA, which targets a protein of an immune checkpoint pathway for reduced expression. In various aspects, the protein of the immune checkpoint pathway is CTLA-4, PD-1, PD-L1, PD-L2, B7-H3, B7-H4, TIGIT, LAG3, CD112 TIM3, BTLA, or co-stimulatory receptor: ICOS, OX40, 41BB, or GITR. The protein of the immune-checkpoint pathway in certain instances is CTLA4, PD-1, PD-L1, B7-H3, B7H4, or TIM3. Immune checkpoint signaling pathways are reviewed in Pardoll, Nature Rev Cancer 12(4): 252-264 (2012).

In exemplary embodiments, the NPs of the present disclosure comprise a mixture of RNA molecules. In exemplary aspects, the mixture of RNA molecules is RNA isolated from cells from a human and optionally, the human has a tumor. In some aspects, the mixture of RNA is RNA isolated from the tumor of the human. In exemplary aspects, the human has cancer, optionally, any cancer described herein. Optionally, the tumor from which RNA is isolated is selected from the group consisting of a glioma, (including, but not limited to, a glioblastoma), a medulloblastoma, a diffuse intrinsic pontine glioma, and a peripheral tumor with metastatic infiltration into the central nervous system (e.g., melanoma or breast cancer). In exemplary aspects, the tumor from which RNA is isolated is a tumor of a cancer, e.g., any of these cancers described herein.

In various aspects, the nucleic acid molecule (e.g., RNA molecule) further comprises a nucleotide sequence encoding a chimeric protein comprising a LAMP protein. In certain aspects, the LAMP protein is a LAMP1, LAMP 2, LAMP3, LAMP4, or LAMP5 protein.

Cores

In exemplary embodiments, the nanoparticles of the present disclosure function as a delivery vehicle for a therapeutic agent or diagnostic agent or a combination thereof. In various aspects, the nanoparticles of the present disclosure function as a delivery vehicle for a theranostic agent, which functions as both a therapeutic agent and a diagnostic agent. In exemplary embodiments, the nanoparticle of the present disclosure comprises a core comprising a therapeutic agent or diagnostic agent or a combination thereof. In exemplary instances, the therapeutic agent is a chemotherapeutic agent or an immunotherapeutic agent. Optionally, the immunotherapeutic agent is a PD-L1 or PD-1 inhibitor. In various aspects, the PD-L1 or PD-1 inhibitor is an antisense oligonucleotide or an siRNA. In various aspects, the diagnostic agent is an imaging agent, such as any one of those described herein. Optionally, the imaging agent comprises iron oxide nanoparticles.

Chemotherapeutic Agents

Chemotherapeutic agents suitable for inclusion in the presently disclosed multilamellar RNA NPs are known in the art, and include, but not limited to, platinum coordination compounds, topoisomerase inhibitors, antibiotics, antimitotic alkaloids and difluoronucleosides, as described in U.S. Pat. No. 6,630,124 (incorporated herein by reference).

In some embodiments, the chemotherapeutic agent is a platinum coordination compound. The term “platinum coordination compound” refers to any tumor cell growth inhibiting compound that provides platinum in the form of an ion. In some embodiments, the platinum coordination compound is cis-diamminediaquoplatinum (II)-ion; chloro(diethylenetriamine)-platinum(II)chloride; dichloro(ethylenediamine)-platinum(II), diammine(1,1-cyclobutanedicarboxylato) platinum(II) (carboplatin); spiroplatin; iproplatin; diammine(2-ethylmalonato)-platinum(II); ethylenediaminemalonatoplatinum(II); aqua(1,2-diaminodyclohexane)-sulfatoplatinum(II); (1,2-diaminocyclohexane)malonatoplatinum(II); (4-caroxyphthalato)(1,2-diaminocyclohexane)platinum(II); (1,2-diaminocyclohexane)-(isocitrato)platinum(II); (1,2-diaminocyclohexane)cis(pyruvato)platinum(II); (1,2-diaminocyclohexane)oxalatoplatinum(II); ormaplatin; or tetraplatin.

In some embodiments, cisplatin is the platinum coordination compound employed in the compositions and methods of the present disclosure. Cisplatin is commercially available under the name PLATINOL™ from Bristol Myers-Squibb Corporation and is available as a powder for constitution with water, sterile saline or other suitable vehicle. Other platinum coordination compounds suitable for use in the context of the present disclosure are known and are available commercially and/or can be prepared by known techniques. Cisplatin, or cis-dichlorodiammineplatinum II, has been used successfully for many years as a chemotherapeutic agent in the treatment of various human solid malignant tumors. More recently, other diamino-platinum complexes have also shown efficacy as chemotherapeutic agents in the treatment of various human solid malignant tumors. Such diamino-platinum complexes include, but are not limited to, spiroplatinum and carboplatinum. Although cisplatin and other diamino-platinum complexes have been widely used as chemotherapeutic agents in humans, they have had to be delivered at high dosage levels that can lead to toxicity problems such as kidney damage.

In some embodiments, the chemotherapeutic agent is a topoisomerase inhibitor. Topoisomerases are enzymes that are capable of altering DNA topology in eukaryotic cells. Topoisomerases are critical for cellular functions and cell proliferation. Generally, there are two classes of topoisomerases in eukaryotic cells, type I and type II. Topoisomerase I is a monomeric enzyme of approximately 100,000 molecular weight. The enzyme binds to DNA and introduces a transient single-strand break, unwinds the double helix (or allows it to unwind), and subsequently reseals the break before dissociating from the DNA strand. Various topoisomerase inhibitors have been shown clinical efficacy in the treatment of humans afflicted with ovarian cancer, breast cancer, esophageal cancer or non-small cell lung carcinoma.

In some aspects, the topoisomerase inhibitor is camptothecin or a camptothecin analog. Camptothecin is a water-insoluble, cytotoxic alkaloid produced by Camptotheca accuminata trees indigenous to China and Nothapodytes foetida trees indigenous to India. Camptothecin inhibits growth of a number of tumor cells. Compounds of the camptothecin analog class are typically specific inhibitors of DNA topoisomerase I. Compounds of the camptothecin analog class include, but are not limited to; topotecan, irinotecan and 9-aminocamptothecin.

In additional embodiments, the chemotherapeutic agent is any tumor cell growth inhibiting camptothecin analog claimed or described in: U.S. Pat. No. 5,004,758 and European Patent Application Number 88311366.4, published as EP 0 321 122; U.S. Pat. No. 4,604,463 and European Patent Application Publication Number EP 0 137 145; U.S. Pat. No. 4,473,692 and European Patent Application Publication Number EP 0 074 256; U.S. Pat. No. 4,545,880 and European Patent Application Publication Number EP 0 074 256; European Patent Application Publication Number EP 0 088 642; Wani et al., J. Med. Chem., 29, 2358-2363 (1986); Nitta et al., Proc. 14th International Congr. Chemotherapy, Kyoto, 1985, Tokyo Press, Anticancer Section 1, p. 28-30; especially a compound called CPT-11. CPT-11 is a camptothecin analog with a 4-(piperidino)-piperidine side chain joined through a carbamate linkage at C-10 of 10-hydroxy-7-ethyl camptothecin. CPT-11 is currently undergoing human clinical trials and is also referred to as irinotecan; Wani et al, J. Med. Chem., 23, 554 (1980); Wani et. al., J. Med. Chem., 30, 1774 (1987); U.S. Pat. No. 4,342,776; U.S. patent application Ser. No. 581,916, filed on Sep. 13, 1990 and European Patent Application Publication Number EP 418 099; U.S. Pat. No. 4,513,138 and European Patent Application Publication Number EP 0 074 770; U.S. Pat. No. 4,399,276 and European Patent Application Publication Number 0 056 692; the entire disclosure of each of which is hereby incorporated by reference. All of the above-listed compounds of the camptothecin analog class are available commercially and/or can be prepared by known techniques including those described in the above-listed references. The topoisomerase inhibitor may be selected from the group consisting of topotecan, irinotecan and 9-aminocamptothecin.

The preparation of numerous compounds of the camptothecin analog class (including pharmaceutically acceptable salts, hydrates and solvates thereof) as well as the preparation of oral and parenteral pharmaceutical compositions comprising such a compounds of the camptothecin analog class and an inert, pharmaceutically acceptable carrier or diluent, is extensively described in U.S. Pat. No. 5,004,758 and European Patent Application Number 88311366.4, published as Publication Number EP 0 321 122, the teachings of which are incorporated herein by reference.

In still yet other embodiments, the chemotherapeutic agent is an antibiotic compound. Suitable antibiotic include, but are not limited to, doxorubicin, mitomycin, bleomycin, daunorubicin and streptozocin.

In some embodiments, the chemotherapeutic agent is an antimitotic alkaloid. In general, antimitotic alkaloids can be extracted from Catharanthus roseus, and have been shown to be efficacious as anticancer chemotherapy agents. A great number of semi-synthetic derivatives have been studied both chemically and pharmacologically (see, O. Van Tellingen et al, Anticancer Research, 12, 1699-1716 (1992)). The antimitotic alkaloids of the present invention include, but are not limited to, vinblastine, vincristine, vindesine, paclitaxel (PTX; Taxol®) and vinorelbine. The latter two antimitotic alkaloids are commercially available from Eli Lilly and Company, and Pierre Fabre Laboratories, respectively (see, U.S. Pat. No. 5,620,985). In an exemplary aspect of the present invention, the antimitotic alkaloid is vinorelbine.

In other embodiments of the invention, the chemotherapeutic agent is a difluoronucleoside. 2-deoxy-2,2-difluoronucleosides are known in the art as having antiviral activity. Such compounds are disclosed and taught in U.S. Pat. Nos. 4,526,988 and 4,808,614. European Patent Application Publication 184,365 discloses that these same difluoronucleosides have oncolytic activity. In certain specific aspects, the 2-deoxy-2,2-difluoronucleoside used in the compositions and methods of the present invention is 2-deoxy-2,2-difluorocytidine hydrochloride, also known as gemcitabine hydrochloride. Gemcitabine is commercially available or can be synthesized in a multi-step process as disclosed and taught in U.S. Pat. Nos. 4,526,988, 4,808,614 and 5,223,608, the teachings of which are incorporated herein by reference.

In exemplary aspects, the chemotherapeutic agent is a hormone therapy agent. In exemplary instances, the hormone therapy agent is, for instance, letrozole, tamoxifen, bazedoxifene, exemestane, leuprolide, goserelin, fulvestrant, anastrozole, or toremifene. In exemplary aspects, the hormone therapy agent is a luteinizing hormone (LH) blocker, e.g., gosarelin, or an LH releasing hormone (RH) agonist. In exemplary aspects, the hormone therapy agent is an ER-targeted agent (e.g., fulvestrant or tamoxifen), rapamycin, a rapamycin analog (e.g., everolimus, temsirolimus, ridaforolimus, zotarolimus, and 32-deoxo-rapamycin), an anti-HER2 drug (e.g., trastuzumab, pertuzumab, lapatinib, T-DM1, or neratinib) or a PI3K inhibitor (e.g., taselisib, alpelisib or buparlisib).

Immunotherapeutic Agents

As used herein, the term “immunotherapeutic agent” refers to any therapeutic agent which boosts the body's natural defenses to fight a disease, e.g., cancer. In various aspects, the immunotherapeutic agent is a cell or a molecule, e.g., a nucleic acid molecule, a protein or peptide. Optionally, the cell is an engineered cell made to express the nucleic acid molecule, protein, or peptide. The immunotherapeutic agent can be, for instance, a monoclonal antibody, an oncolytic virus therapeutic agent, a T-cell therapeutic agent, or a cancer vaccine. The monoclonal antibody may be, e.g., ipilimumab, nivolumab, pembrolizumab, atezolizumab, avelumab, or durvalumab. In various instances, the immunotherapeutic agent is a CAR T cell therapeutic agent, e.g., tisagenlecleucel, axicabtagene, or ciloleucel. In various aspects, the immunotherapeutic agent is a tumor-agnostic agent, e.g., lacrotrectinib. In various aspects, the immunotherapeutic agent is a cytokine, optionally, an interferon or an interleukin. In various aspects, the cytokine is IFN-alpha (Roferon-A [2a], Intron A [2b], Alferon [2a]) or IL-2 (aldesleukin)).

In exemplary aspects, the therapeutic agents comprise or are nucleic acids. Optionally, the therapeutic agents are antisense oligonucleotides (ASOs) or siRNAs. In various instances, the ASOs or siRNAs are not the same nucleic acids present in the alternating nucleic acid layers—cationic lipid bilayers. In exemplary instances, the ASOs or siRNAs are the same nucleic acids present in the alternating nucleic acid layers—cationic lipid bilayers. In exemplary instances, the ASO or siRNA targets a protein that functions in an immune checkpoint pathway. In exemplary instances, the ASO or siRNA reduces expression of the protein that functions in the immune checkpoint pathway. In various aspects, the protein that functions in the immune checkpoint pathway is one of PD-1, PD-L1, CTLA-4, CTLA-4, PD-1, PD-L1, PD-L2, B7-H3, B7-H4, CEACAM-1, TIGIT, LAG3, CD112, CD112R, CD96, TIM3, BTLA, ICOS, OX40, 41BB, CD27, or GITR.

Imaging Agents

Multifunctional RNA-loaded magnetic liposomes to initiate potent antitumor immunity and function as an early MRI-based imaging biomarker of treatment response was designed and shown to activate dendritic cells (DCs) more effectively than electroporation leading to superior inhibition of tumor growth in treatment models. Inclusion of iron oxide enhanced DC transfection and enabled tracking of DC migration with MRI. It was shown that T2*-weighted MRI hypointensity in lymph nodes was a strong correlate of DC trafficking and suggest that T2*-weighted MRI hypointensity in lymph nodes can be an early predictor of antitumor response. In preclinical tumor models, MRI-predicted “responders” identified two days after vaccination had significantly smaller tumors 2-5 weeks after treatment and lived 100% longer than MRI-predicted “non-responders.” These studies therefore provided a simple, scalable nanoparticle formulation to generate robust antitumor immune responses and predict individual treatment outcome with MRI. Without being bound to a particular theory, the multilamellar RNA NPs of the present disclosure comprising iron oxide nanoparticles may be used to activate DCs, inhibit tumor growth, enhance DC transfection and enable tracking of DC migration with MRI. Therefore, the present disclosure further provides a nanoparticle comprising a positively-charged surface and an interior comprising (i) a core and (ii) at least two nucleic acid layers, wherein each nucleic acid layer is positioned between a cationic lipid bilayer, wherein the core comprises a diagnostic agent, such as an imaging agent (e.g., a contrast agent), optionally, gadolinium, a perfluorocarbon microbubble, iron oxide nanoparticle, colloidal gold or gold nanoparticle (see, e.g., Mahan and Doiron, J Nanomaterials, volume 2018, article ID 5837276). In various aspects, the core comprises a radiopharmaceutical (e.g., carbon-11, fluorine-18, gallium-67 or -68, indium-111, iodine-123, -125, -131, krypton-81m, lutetium-177, nitrogen-13, oxygen-15, phosphorus-32, selenium-75, technetium-99m, thallium-201, xenon-133, yttrium-90). In various aspects, the core comprises iron oxide nanoparticles (IONPs) which are useful for imaging tissue or cells via, e.g., magnetic resonance imaging (MRI). In various aspects, the IONPs are Combidex®, Resovist®, Endorem®, or Sinerem®. Optionally, the IONPs are coated with a fatty acid, e.g., a C8-C30 fatty acid. In various aspects, the fatty acid is stearic acid, palmitic acid, myristic acid, lauric acid, capric acid, caprylic acid, palmitoleic acid, cis-vaccenic acid, or oleic acid. In various aspects, the core comprises a plurality of IONPs (optionally wherein each IONP is coated with oleic acid) wherein the plurality is held together by a lipid, e.g., a cationic lipid. Optionally, the plurality of IONPs (optionally coated with oleic acid) are held together by DOTAP. Methods of making such IONPs held together by a DOTAP coating are described herein.

Methods of Manufacture

The present disclosure also provides a method of making a nanoparticle comprising a positively-charged surface and an interior comprising (i) a core and (ii) at least two nucleic acid layers, wherein each nucleic acid layer is positioned between a cationic lipid bilayer, said method comprising: (A) mixing nucleic acid molecules and liposomes at a RNA:liposome ratio of about 1 to about 5 to about 1 to about 25, such as about 1 to about 5 to about 1 to about 20, optionally, about 1 to about 15, to obtain a RNA-coated liposomes, wherein the liposomes are made by a process of making liposomes comprising drying a lipid mixture comprising a cationic lipid and an organic solvent by evaporating the organic solvent under a vacuum, and (B) mixing the RNA-coated liposomes with a surplus amount of liposomes.

In exemplary aspects, the nanoparticle made by the presently disclosed method accords with the descriptions of the presently disclosed nanoparticles described herein. For example, the nanoparticle made by the presently disclosed methods has a zeta potential of about +40 mV to about +60 mV, optionally, about +45 mV to about +55 mV. Optionally, the zeta potential of the nanoparticle made by the presently disclosed methods is about +50 mV. In various aspects, the core of the nanoparticle made by the presently disclosed methods comprises less than about 0.5 wt % nucleic acid and/or the core comprises a cationic lipid bilayer and/or the outermost layer of the nanoparticle comprises a cationic lipid bilayer and/or the surface of the nanoparticle comprises a plurality of hydrophilic moieties of the cationic lipid of the cationic lipid bilayer.

In exemplary aspects, the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent. In various instances, the process of making liposomes further comprises rehydrating the lipid mixture with a rehydration solution to form a rehydrated lipid mixture and then agitating, resting, and sizing the rehydrated lipid mixture. Optionally, sizing the rehydrated lipid mixture comprises sonicating, extruding and/or filtering the rehydrated lipid mixture.

A description of an exemplary method of making a nanoparticle comprising a positively-charged surface and an interior comprising (i) a core and (ii) at least two nucleic acid layers, wherein each nucleic acid layer is positioned between a cationic lipid bilayer is provided herein at Example 1. Any one or more of the steps described in Example 1 may be included in the presently disclosed method. For instance, in some embodiments, the method comprises one or more steps required for preparing the RNA prior to being complexed with the liposomes. In exemplary aspects, the method comprises downstream steps to prepare the nanoparticles for administration to a subject, e.g., a human. In exemplary instances, the method comprises formulating the NP for intravenous injection. The method comprises in various aspects adding one or more pharmaceutically acceptable carriers, diluents, or excipients, and optionally comprises packaging the resulting composition in a container, e.g., a vial, a syringe, a bag, an ampoule, and the like. The container in some aspects is a ready-to-use container and optionally is for single-use.

Further provided herein are nanoparticles made by the presently disclosed method of making a nanoparticle.

Cells and Populations Thereof

Additionally provided herein is a cell comprising (e.g., transfected with) a nanoparticle of the present disclosure. In exemplary aspects, the cell is any type of cell that can contain the presently disclosed nanoparticle. The cell in some aspects is a eukaryotic cell, e.g., plant, animal, fungi, or algae. In alternative aspects, the cell is a prokaryotic cell, e.g., bacteria or protozoa. In exemplary aspects, the cell is a cultured cell. In alternative aspects, the cell is a primary cell, i.e., isolated directly from an organism, e.g., a human. The cell may be an adherent cell or a suspended cell, i.e., a cell that grows in suspension. The cell in exemplar aspects is a mammalian cell. Most preferably, the cell is a human cell. The cell can be of any cell type, can originate from any type of tissue, and can be of any developmental stage. In exemplary aspects, the cell comprising the liposome is an antigen presenting cell (APC). As used herein, “antigen presenting cell” or “APC” refers to an immune cell that mediates the cellular immune response by processing and presenting antigens for recognition by certain T cells. In exemplary aspects, the APC is a dendritic cell, macrophage, Langerhans cell or a B cell. In exemplary aspects, the APC is a dendritic cell (DC). In exemplary aspects, when the cells are administered to a subject, e.g., a human, the cells are autologous to the subject. In exemplary instances, the immune cell is a tumor associated macrophage (TAM).

Also provided by the present disclosure is a population of cells wherein at least 50%, at least 60%, at least 70%, at least 80%, at least 90%, or at least 95% of the population are cells comprising (e.g., transfected with) a nanoparticle of the present disclosure. The population of cells in some aspects is heterogeneous cell population or, alternatively, in some aspects, is a substantially homogeneous population, in which the population comprises mainly cells comprising a nanoparticle of the present disclosure.

Pharmaceutical Compositions

Provided herein are compositions comprising a nanoparticle of the present disclosure, a cell comprising the nanoparticle of the present disclosure, a population of cells of the present disclosure, or any combination thereof, and a pharmaceutically acceptable carrier, excipient or diluent. In exemplary aspects, the composition is a pharmaceutical composition comprising a plurality of nanoparticles according to the present disclosure and a pharmaceutically acceptable carrier, diluent, or excipient and intended for administration to a human. In exemplary aspects, the composition is a sterile composition. In exemplary instances, the composition comprises a plurality of nanoparticles of the present disclosure. Optionally, at least 50% of the nanoparticles of the plurality have a diameter between about 100 nm to about 250 nm. In various aspects, the composition comprises about 10¹⁰ nanoparticles per mL to about 10¹⁵ nanoparticles per mL, optionally about 10¹² nanoparticles ±10% per mL.

In exemplary aspects, the composition of the present disclosure may comprise additional components other than the nanoparticle, cell comprising the nanoparticle, or population of cells. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The composition of the present disclosure can be suitable for administration by any acceptable route, including parenteral and subcutaneous. Other routes include intravenous, intradermal, intramuscular, intraperitoneal, intranodal and intrasplenic, for example. In exemplary aspects, when the composition comprises the liposomes (not cells comprising the liposomes), the composition is suitable for systemic (e.g., intravenous) administration.

If the composition is in a form intended for administration to a subject, it can be made to be isotonic with the intended site of administration. For example, if the solution is in a form intended for administration parenterally, it can be isotonic with blood. The composition typically is sterile. In certain embodiments, this may be accomplished by filtration through sterile filtration membranes. In certain embodiments, parenteral compositions generally are placed into a container having a sterile access port, for example, an intravenous solution bag, or vial having a stopper pierceable by a hypodermic injection needle, or a prefilled syringe. In certain embodiments, the composition may be stored either in a ready-to-use form or in a form (e.g., lyophilized) that is reconstituted or diluted prior to administration.

Use

Without being bound to any particular theory, the data provided herein for the first time support the use of the presently disclosed RNA NPs for increasing an immune response, including inducing an immune response against a tumor in a subject. Accordingly, a method of increasing an immune response against a tumor in a subject is provided by the present disclosure. In exemplary embodiments, the method comprises administering to the subject the pharmaceutical composition of the present disclosure. In exemplary aspects, the nucleic acid molecules are mRNA. Optionally, the composition is systemically administered to the subject. For example, the composition is administered intravenously. In various aspects, the pharmaceutical composition is administered in an amount which is effective to activate dendritic cells (DCs) in the subject. In various instances, the immune response is a T cell-mediated immune response. Optionally, the T cell-mediated immune response comprises activity by tumor infiltrating lymphocytes (TILs). In exemplary aspects, the immune response is the innate immune response.

Also the data provided herein for the first time support the use of the presently disclosed RNA NPs for increasing Dendritic Cell (DC) activation in a subject. A method of activating DCs or increasing DC activation in a subject is accordingly furthermore provided. In exemplary embodiments, the method comprises administering to the subject the pharmaceutical composition of the present disclosure. In exemplary aspects, the nucleic acid molecules are mRNA. Optionally, the composition is systemically administered to the subject. For example, the composition is administered intravenously. In various aspects, the pharmaceutical composition is administered in an amount which is effective to increase an immune response against a tumor in the subject. In various instances, the immune response is a T cell-mediated immune response. Optionally, the T cell-mediated immune response comprises activity by tumor infiltrating lymphocytes (TILs). In exemplary aspects, the immune response is the innate immune response.

As used herein, the term “increase” and words stemming therefrom may not be a 100% or complete increase. Rather, there are varying degrees of increasing of which one of ordinary skill in the art recognizes as having a potential benefit or therapeutic effect. In exemplary embodiments, the increase provided by the methods is at least or about a 10% increase (e.g., at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase).

The present disclosure also provides a method of delivering RNA molecules to an intra-tumoral microenvironment, lymph node, and/or a reticuloendothelial organ. In exemplary embodiments, the method comprises administering to the subject a presently disclosed pharmaceutical composition. Optionally, the reticuloendothelial organ is a spleen or liver. Provided herein are methods of delivery RNA to cells of a tumor, e.g., a brain tumor, comprising systemically (e.g., intravenously) administering a presently disclosed composition, wherein the composition comprises the nanoparticles. Also provided herein are methods of delivering RNA to cells in a microenvironment of a tumor, optionally a brain tumor. In exemplary embodiments, the method comprises systemically (e.g., intravenously) administering a presently disclosed composition, wherein the composition comprises the nanoparticle. In some aspects, the nanoparticle comprises an siRNA targeting a protein of an immune checkpoint pathway, optionally, PD-L1. In various aspects, the cells in the microenvironment are antigen-presenting cells (APCs), optionally, tumor associated macrophages. The present disclosure also provides methods of activating antigen-presenting cells in a tumor microenvironment. In exemplary embodiments, the method comprises systemically (e.g., intravenously) administering a presently disclosed composition, wherein the composition comprises the NP.

The present disclosure provides methods of delivering RNA molecules to cells. In exemplary embodiments, the method comprises incubating the cells with the NPs of the present disclosure. In exemplary instances, the cells are antigen-presenting cells (APCs), optionally, dendritic cells (DCs). In various instances, the APCs (e.g., DCs) are obtained from a subject. In certain aspects, the RNA molecules are isolated from tumor cells obtained from a subject, e.g., a human. In certain aspects, the RNA molecules are antisense molecules that target a protein of interest for reduced expression. In exemplary aspects, the RNA molecules are siRNA molecules that target a protein of the immune checkpoint pathway. Suitable proteins of the immune checkpoint pathway are known in the art and also described herein. In various instances, the siRNA target PD-L1.

Once RNA has been delivered to the cells, if the delivery is in vitro or ex vivo, the cells may be administered to a subject for treatment of a disease. Accordingly, the present disclosure provides a method of treating a subject with a disease. In exemplary embodiments, the method comprises delivering RNA molecules to cells of the subject in accordance with the above-described method of delivering RNA molecules to cells. In some aspects, RNA molecules are delivered to the cells ex vivo and the cells are administered to the subject. Alternatively, the method comprises administering the liposomes directly to the subject. In exemplary embodiments, the method of treating a subject with a disease comprises administering a composition of the present disclosure in an amount effective to treat the disease in the subject. In exemplary aspects, the disease is cancer, and, in some aspects, the cancer is located across the blood brain barrier and/or the subject has a tumor located in the brain. In some aspects, the tumor is a glioma, a low grade glioma or a high grade glioma, specifically a grade III astrocytoma or a glioblastoma. Alternatively, the tumor could be a medulloblastoma or a diffuse intrinsic pontine glioma. In another example, the tumor could be a metastatic infiltration from a non-CNS tumor, e.g., breast cancer, melanoma, or lung cancer. In exemplary aspects, the composition comprises the liposomes, and optionally, the composition comprising the liposomes are intravenously administered to the subject. In alternative aspects, the composition comprises cells transfected with the liposome. Optionally, the cells of the composition are APCs, optionally, DCs. In exemplary aspects, the composition comprising the cells comprising the liposome is intradermally administered to the subject, optionally, wherein the composition is intradermally administered to the groin of the subject. In exemplary instances, the DCs are isolated from white blood cells (WBCs) obtained from the subject, optionally, wherein the WBCs are obtained via leukapheresis. In some aspects, the RNA molecules encode a tumor antigen. In some aspects, the RNA molecules are isolated from tumor cells, e.g., tumor cells are cells of a tumor of the subject. Accordingly, a method of treating a subject with a disease is furthermore provided herein. In exemplary embodiments, the method comprises delivering RNA molecules to cells of the subject according to the presently disclosed method of delivering RNA molecules to an intra-tumoral microenvironment, lymph node, and/or a reticuloendothelial organ. In various aspects, RNA molecules are ex vivo delivered to the cells and the cells are administered to the subject. In exemplary embodiments, the method comprises administering to the subject a pharmaceutical composition of the present disclosure in an amount effective to treat the disease in the subject. In various instances, the subject has a cancer or a tumor, optionally, a malignant brain tumor, optionally, a glioblastoma, medulloblastoma, diffuse intrinsic pontine glioma, or a peripheral tumor with metastatic infiltration into the central nervous system.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a disease of the present disclosure can provide any amount or any level of treatment. Furthermore, the treatment provided by the method may include treatment of one or more conditions or symptoms or signs of the disease being treated. For instance, the treatment method of the presently disclosure may inhibit one or more symptoms of the disease. Also, the treatment provided by the methods of the present disclosure may encompass slowing the progression of the disease. The term “treat” also encompasses prophylactic treatment of the disease. Accordingly, the treatment provided by the presently disclosed method may delay the onset or reoccurrence/relapse of the disease being prophylactically treated. In exemplary aspects, the method delays the onset of the disease by 1 day, 2 days, 4 days, 6 days, 8 days, 10 days, 15 days, 30 days, two months, 4 months, 6 months, 1 year, 2 years, 4 years, or more. The prophylactic treatment encompasses reducing the risk of the disease being treated. In exemplary aspects, the method reduces the risk of the disease 2-fold, 5-fold, 10-fold, 20-fold, 50-fold, 100-fold, or more.

In certain aspects, the method of treating the disease may be regarded as a method of inhibiting the disease, or a symptom thereof. As used herein, the term “inhibit” and words stemming therefrom may not be a 100% or complete inhibition or abrogation. Rather, there are varying degrees of inhibition of which one of ordinary skill in the art recognizes as having a potential benefit or therapeutic effect. The presently disclosed methods may inhibit the onset or re-occurrence of the disease or a symptom thereof to any amount or level. In exemplary embodiments, the inhibition provided by the methods is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition).

With regard to the foregoing methods, the NPs or the composition comprising the same in some aspects is systemically administered to the subject. Optionally, the method comprises administration of the liposomes or composition by way of parenteral administration. In various instances, the liposome or composition is administered to the subject intravenously.

In various aspects, the NP or composition is administered according to any regimen including, for example, daily (1 time per day, 2 times per day, 3 times per day, 4 times per day, 5 times per day, 6 times per day), three times a week, twice a week, every two days, every three days, every four days, every five days, every six days, weekly, bi-weekly, every three weeks, monthly, or bi-monthly. In various aspects, the liposomes or composition is/are administered to the subject once a week.

Subjects

The subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human is a child aged 17 years or less. In exemplary aspects, the subject has a DMG. In various instances, the DMG is diffuse intrinsic pontine glioma (DIPG).

Cancer

The cancer treatable by the methods disclosed herein may be any cancer, e.g., any malignant growth or tumor caused by abnormal and uncontrolled cell division that may spread to other parts of the body through the lymphatic system or the blood stream.

The cancer in some aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, or lung cancer, e.g., non-small cell lung cancer (NSCLC), or bronchioloalveolar carcinoma.

The following examples are given merely to illustrate the present invention and not in any way to limit its scope.

### EXAMPLES

### Example 1

This example describes a method of making nanoparticles of the present disclosure.

### Preparation of DO TAP Liposomes

On Day 1, the following steps were carried out in the fume hood. Water was added to a rotavapor bath. Chloroform (20 mL) was poured into a sterile, glass graduated cylinder. After opening a vial containing 1 g of DOTAP, 5 mL chloroform was added to the DOTAP vial using a glass pipette. The volume of chloroform and DOTAP was then transferred into a 1-L evaporating flask. The DOTAP vial was washed by adding a second 5-mL volume of chloroform to the DOTAP vial to dissolve any remaining DOTAP in the vial and then transferring this volume of chloroform from the DOTAP vial to the evaporating flask. This washing step was repeated 2 more times until all the chloroform in the graduated cylinder was used. The evaporating flask was then placed into the Buchi rotavapor. The water bath was turned on and adjusted to 25° C. The evaporating flask was moved downward until it touched the water bath. The rotation speed of the rotavapor was adjusted to 2. The vacuum system was turned on and adjusted to 40 mbar. After 10 minutes, the vacuum system was turned off and the chloroform was collected from the collector flask. The amount of chloroform collected was measured. Once the collector flask is repositioned, the vacuum was turned on again and the contents in the evaporating flask was allowed to dry overnight until the chloroform was completely evaporated.

On Day 2, using a sterile graduated cylinder, PBS (200 mL) was added to a new, sterile 500-mL PBS bottle maintained at room temperature. A second 500-mL PBS bottle was prepared for collecting DOTAP. The Buchi rotavapor water bath was set to 50° C. PBS (50 mL) was added into the evaporating flask using a 25-mL disposable serological pipette. The evaporating flask was positioned in the Buchi rotavapor and moved downward until ⅓ of the flask was submerged into the water bath. The rotation speed of the rotavapor was set to 2, allowed to rotate for 10 min, and then rotation was turned off. A 50-mL volume of PBS with DOTAP from the evaporating flask was transferred to the second 500 mL PBS bottle. The steps were repeated (3-times) until the entire volume of PBS in the PBS bottle was used. The final volume of the second 500 mL PBS bottle was 400 mL. The lipid solution in the second 500 mL PBS bottle was vortexed for 30 s and then incubated at 50° C. for 1 hour. During the 1 hour incubation, the bottle was vortexed every 10 min. The second 500 mL PBS bottle was allowed to rest on overnight at room temperature.

On Day 3, PBS (200 mL) was added to the second 500 mL PBS bottle containing DOTAP and PBS. The second 500 mL PBS bottle was placed into an ultrasonic bath. Water was filled in the ultrasonic bath and the second 500 mL PBS bottle was sonicated for 5 min. The extruder was washed with PBS (100 mL) and this wash step was repeated. A 0.45 μm pore filter was assembled into a filtration unit and a new (third) 500 mL PBS bottle was positioned into the output tube of the extruder. In a biological safety cabinet, the DOTAP-PBS mixture was loaded into the extruder, until about 70% of the third PBS bottle was filled. The extruder was then turned on and the DOTAP PBS mixture was added until all the mixture was run through the extruder. Subsequently, a 0.22 μm pore filter was assembled into the filtration unit and a new (third) 500 mL PBS bottle was positioned into the output tube of the extruder. The previously filtered DOTAP-PBS mixture was loaded and run again throughout. The samples comprising DOTAP lipid nanoparticles (NPs) in PBS were then stored at 4° C.

### RNA Preparation

Prior to incorporation into NPs, RNA was prepared in one of a few ways. Total tumor RNA was prepared by isolating total RNA (including rRNA, tRNA, mRNA) from tumor cells. In vitro transcribed mRNA was prepared by carrying out in vitro transcription reactions using cDNA templates produced by reverse transcription of total tumor RNA. Tumor antigen-specific and non-specific RNAs were either made in-house or purchased from a vendor.

Total Tumor RNA: Total tumor-derived RNA from tumor cells (e.g., B16F0, B16F10, and KR158-luc) is isolated using commercially available RNeasy mini kits (Qiagen) based on manufacturer instructions.

In vitro transcribed mRNA: Briefly, RNA is isolated using commercially available RNeasy mini kits (Qiagen) per manufacturer's instructions and cDNA libraries were generated by RT-PCR. Using a SMARTScribe Reverse Transcriptase kit (Takara), a reverse transcriptase reaction by PCR was performed on the total tumor RNA in order to generate cDNA libraries. The resulting cDNA was then amplified using Takara Advantage 2 Polymerase mix with T7/SMART and CDS III primers, with the total number of amplification cycles determined by gel electrophoresis. Purification of the cDNA was performed using a Qiagen PCR purification kit per manufacturer's instructions. In order to isolate sufficient mRNA for use in each RNA-nanoparticle vaccine, mMESAGE mMACHINE (Invitrogen) kits with T7 enzyme mix were used to perform overnight in vitro transcription on the cDNA libraries. Housekeeping genes were assessed to ensure fidelity of transcription. The resulting mRNA was then purified with a Qiagen RNeasy Maxi kit to obtain the final mRNA product.

Tumor Antigen-Specific and Non-Specific mRNA:

Plasmids comprising DNA encoding tumor antigen-specific RNA (RNA encoding, e.g., pp65, OVA) and non-specific RNA (RNA encoding, e.g., Green Fluorescent Protein (GFP), luciferase) are linearized using restriction enzymes (i.e., SpeI) and purified with Qiagen PCR MiniElute kits. Linearized DNA is subsequently transcribed using the mmRNA in vitro transcription kit (Life technologies, Invitrogen) and cleaned up using RNA Maxi kits (Qiagen). In alternative methods, non-specific RNA is purchased from Trilink Biotechnologies (San Diego, Calif.).

### Preparation of Multilamellar RNA Nanoparticles (NPs)

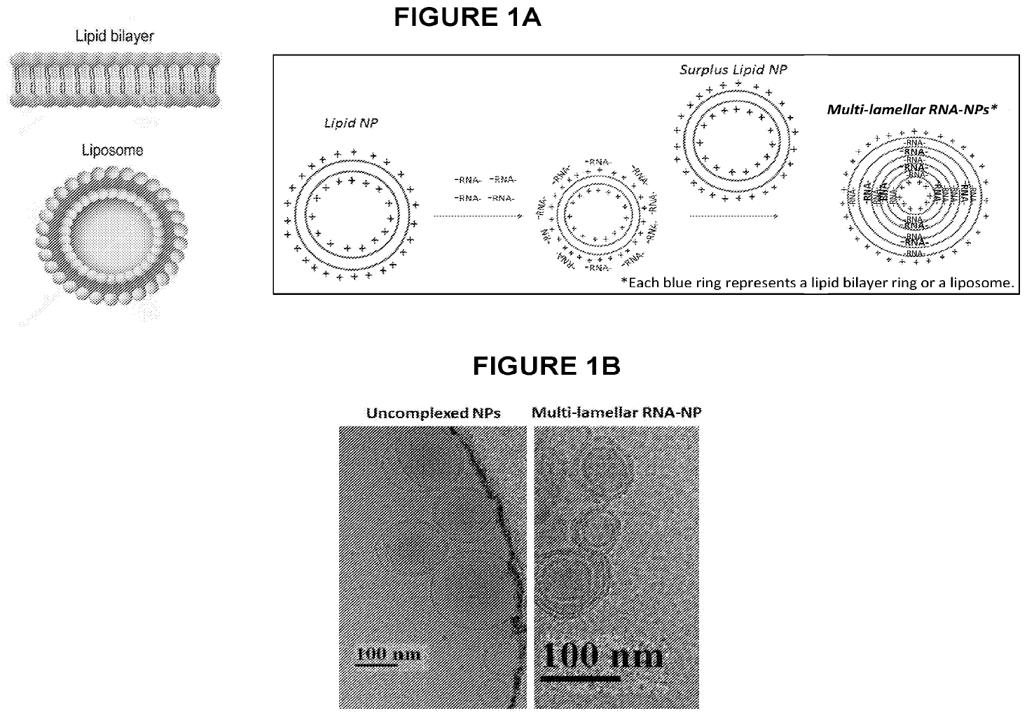

The DOTAP lipid NPs were complexed with RNA to make multilamellar RNA-NPs which were designed to have several layers of mRNA contained inside a tightly coiled liposome with a positively charged surface and an empty core (FIG. 1A). Briefly, in a safety cabinet, RNA was thawed from −80° C. and then placed on ice, and samples comprising PBS and DOTAP (e.g., DOTAP lipid NPs) were brought up to room temperature. Once components were prepared, the desired amount of RNA was mixed with PBS in a sterile tube. To the sterile tube containing the mixture of RNA and PBS, the appropriate amount of DOTAP lipid NPs was added without any physical mixing (without e.g., inversion of the tube, without vortexing, without agitation). The mixture of RNA, PBS, and DOTAP was incubated for about 15 minutes to allow multilamellar RNA-NP formation. After 15 min, the mixture was gently mixed by repeatedly inverting the tube. The mixture was then considered ready for systemic (i.e., intravenous) administration.

The amount of RNA and DOTAP lipid NPs (liposomes) used in the above preparation is pre-determined or pre-selected. In some instances, a ratio of about 15 μg liposomes per about 1 μg RNA were used. For instance, about 75 μg liposomes are used per ˜5 μg RNA or about 375 μg liposomes are used per ˜25 μg RNA. In other instances, about 7.5 μg liposomes were used per 1 μg RNA. Thus, in exemplary instances, about 1 μg to about 20 μg liposomes are used for every μg RNA used.

### Example 2

This example describes the characterization of the nanoparticles of the present disclosure.

Cryo-Electron Microscopy (CEM)

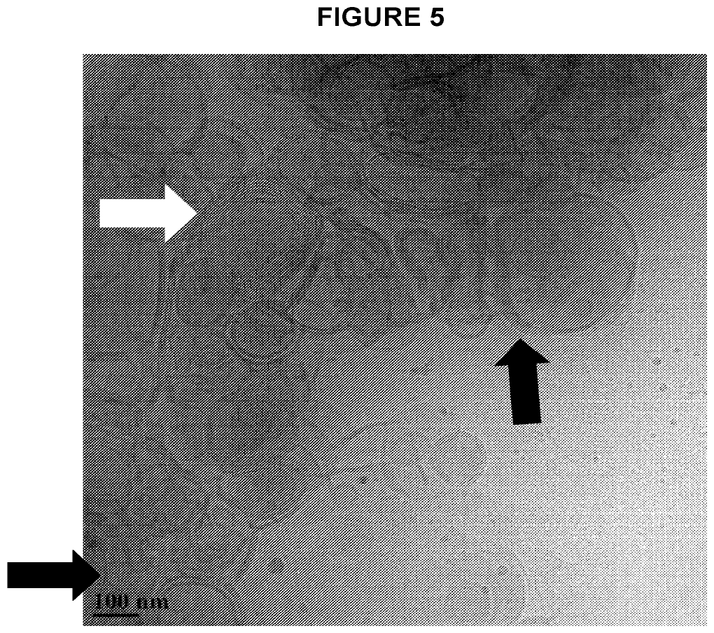

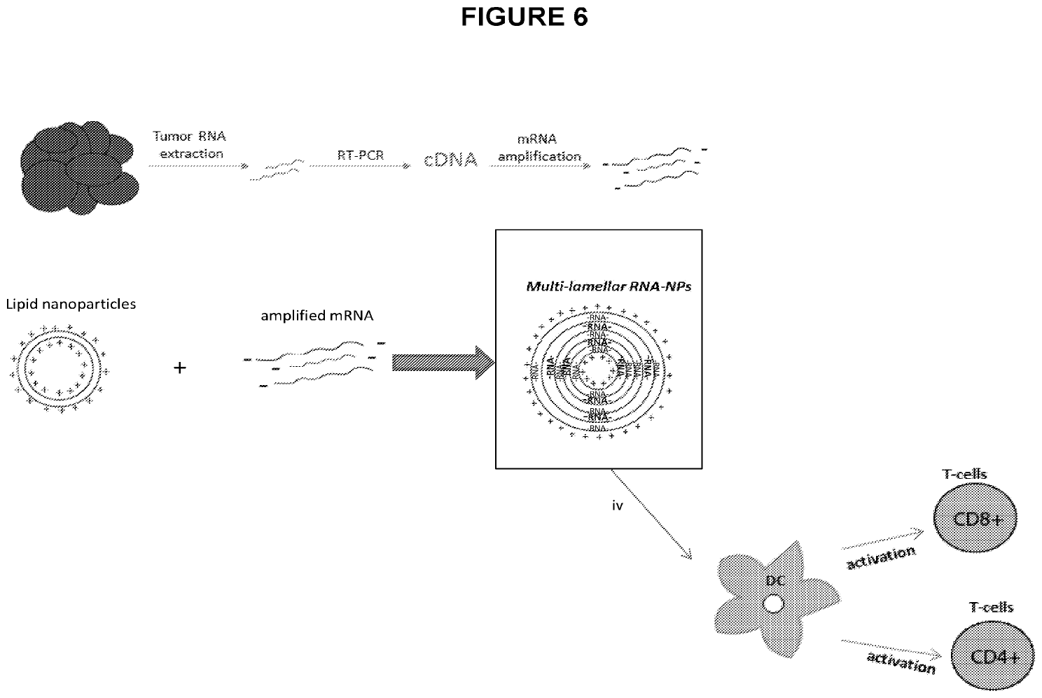

CEM was used to analyze the structure of multilamellar RNA-NPs prepared as described in Example 1 and control NPs devoid of RNA (uncomplexed NPs) which were made by following all the steps of Example 1, except for the steps under “RNA Preparation” and “Preparation of Multilamellar RNA nanoparticles (NPs)”. CEM was carried out as essentially described in Sayour et al., Nano Lett 17(3) 1326-1335 (2016). Briefly, samples comprising multilamellar RNA-NPs or control NPs were kept on ice prior to being loaded in a snap-freezed in Vitrobot (and automated plunge-freezer for cryoTEM, that freezes samples without ice crystal formation, by controlling temperature, relative humidity, blotting conditions and freezing velocity). Samples were then imaged in a Tecnai G2 F20 TWIN 200 kV/FEG transmission electron microscope with a Gatan UltraScan 4000 (4k×4k) CCD camera. The resulting CEM images are shown in FIG. 1B. The right panel is a CEM image of multilamellar RNA-NPs and the left panel is a CEM image of control NPs (uncomplexed NPs). As shown in FIG. 1B, the control NPs contained at most 2 layers, whereas multilamellar RNA NPs contained several layers. FIG. 5 provides another CEM image of exemplary multilamellar RNA NPs. Here, the multiple layers of RNA layers alternating with lipid layers are especially evident.

Zeta Potentials

Zeta potentials of multilamellar RNA NPs were measured by phase analysis light scattering (PALS) using a Brookhaven ZetaPlus instrument (Brookhaven Instruments Corporation, Holtsville, N.Y.), as essentially described in Sayour et al., Nano Lett 17(3) 1326-1335 (2016). Briefly, uncomplexed NPs or RNA-NPs (200 μL) were resuspended in PBS (1.2 mL) and loaded in the instrument. The samples were run at 5 runs per sample, 25 cycles each run, and using the Smoluchowski model.

The zeta potential of the multilamellar RNA NPs prepared as described in Example 1 was measured at about +50 mV. Interestingly, this zeta potential of the multilamellar RNA NPs was much higher than those described in Sayour et al., Oncoimmunology 6(1): e1256527 (2016), which measured at around +27 mV. Without being bound to any particular theory, the way in which the DOTAP lipid NPs are made for use in making the multilamellar RNA NPs (Example 1) involving a vacuum-seal method for evaporating off chloroform leads to less environmental oxidation of the DOTAP lipid NPs, which, in turn, may allow for a greater amount of RNA to complex with the DOTAP NPs and/or greater incorporation of RNA into the DOTAP lipid NPs.

RNA Incorporation by Gel Electrophoresis:

A gel electrophoresis experiment was conducted to measure the amount of RNA incorporated into ML liposomes. Based on this experiment, it was qualitatively shown that nearly all, if not all, of the RNA used in the procedure described in Example 1 was incorporated into the DOTAP lipid NPs. Additional experiments to characterize the extent of RNA incorporation are carried out by measuring RNA-NP density and comparing this parameter to that of lipoplexes.

### Example 3

This example demonstrates the in vivo sites of localization of RNA-NPs upon systemic administration and that RNA NPs mediate peripheral and intratumoral activation of DCs.

DOTAP lipid NPs made as essentially described in Example 1 are complexed with Cre recombinase-encoding mRNA to make Cre-encoding RNA-NPs. These multilamellar RNA-NPs are administered to Ai14 transgenic mice, which carry a STOP cassette flanked by loxP. The STOP cassette prevents the transcription of tdTomato until Cre-recombinase is expressed. A week after RNA-NPs are administered, the lymph nodes, spleens and livers of the transgenic mice are harvested, sectioned and stained with DAPI. The expression of tdTomato is analyzed by fluorescent microscopy following the procedures as essentially described in Sayour et al, Nano Letters 2018. It is expected that the Cre-mRNA-NPs localize in vivo to lymphoid organs, including liver, spleen, and lymph nodes.

DOTAP lipid NPs made as essentially described in Example 1 are complexed with non-specific RNA (e.g., RNA that was not tumor antigen-specific; ovalbumin (OVA) mRNA) and intravenously injected into C57Bl/6 mice (n=3-4/group) bearing subcutaneous B16F10 tumors. Lymph nodes, spleens, livers, bone marrow and tumors are harvested within 24 hrs and analyzed for expression of the Dendritic Cell (DC) activation marker, CD86, by CD11c cells (*p<0.05 Mann-Whitney) test). It is expected that the OVA mRNA-NPs demonstrate widespread in vivo localization to the lymph nodes, spleens, livers, bone marrow, and tumors and activated the DCs therein (as shown by the increased expression of the activation marker CD86 on CD11c+ cells). Because activated DCs prime antigen-specific T cell responses, lead to anti-tumor efficacy (with increased TILs) in several tumor models, we tested the anti-tumor efficacy of the multi-lamellar RNA NPs.

### Example 4

This example describes a comparison of the nanoparticles of the present disclosure to cationic RNA lipoplexes and anionic RNA lipoplexes.

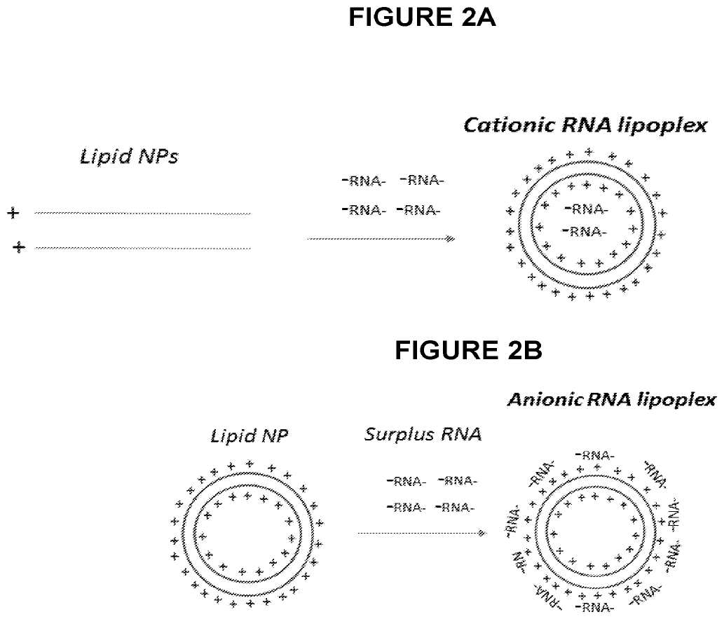

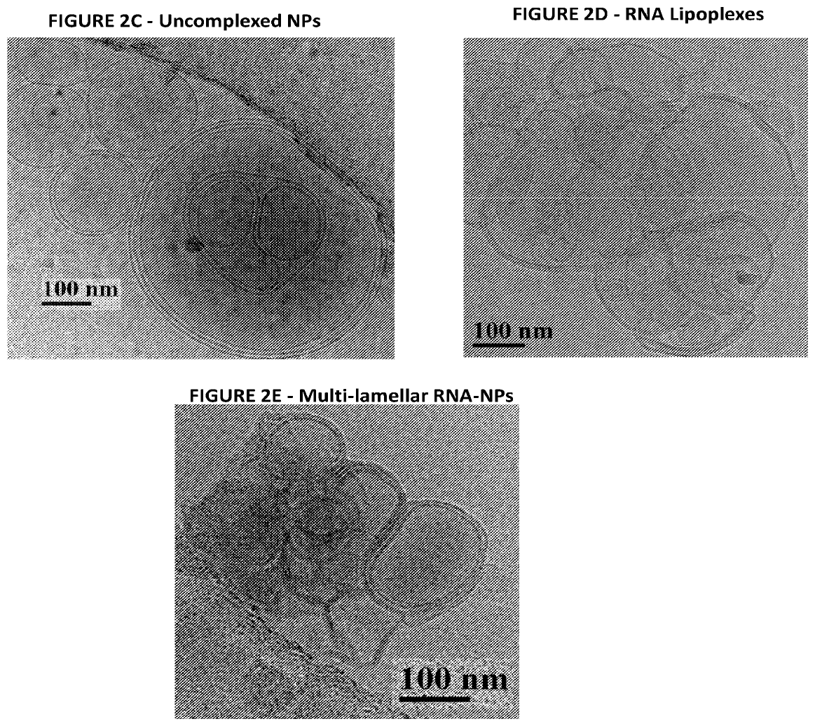

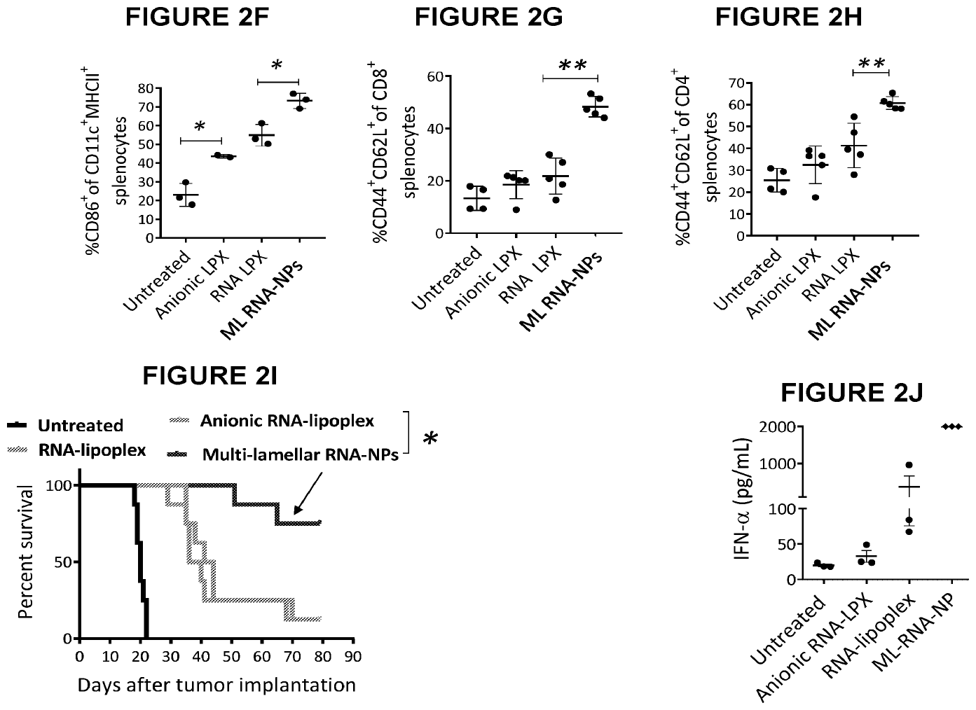

Cationic lipoplexes (LPX) were first developed with mRNA in the lipid core shielded by a net positive charge located on the outer surface (FIG. 2A). Anionic RNA lipoplexes (FIG. 2B) have been developed with an excess of RNA tethered to the surface of bi-lamellar liposomes. RNA-LPX were made by mixing RNA and lipid NP at ratios to equalize charge. Anionic RNA-NPs were made by mixing RNA and lipid NP at ratios to oversaturate lipid NPs with negative charge. Various aspects of the RNA-LPX and anionic RNA LPX were then compared to the multilamellar RNA NPs described in the above examples.

Cryo-Electron Microscopy (CEM) was used to compare the structures of the RNA LPX and the multilamellar RNA-NPs prepared as described in Example 1. Uncomplexed NPs were used as a control. CEM was carried out as essentially described in Example 2. FIG. 2C is a CEM image of uncomplexed NPs, FIG. 2D is a CEM image of RNA LPXs (wherein that mass ratio of liposome to RNA is 3.75:1) and FIG. 2E is a CEM image of the multilamellar RNA-NPs (wherein that mass ratio of liposome to RNA is 15:1). These data support that more RNA is held by the ML RNA-NPs. Additional data show that the concentration drops more with ML RNA-NP complexation versus RNA LPX supporting multilamellar formation of ML RNA-NPs not observed by simple mixing of equivalent amounts of RNA and lipid NPs by mass or charge (i.e., RNA-LPX and anionic RNA-LPX respectively). This supports that more RNA is “held” by ML RNA-NPs.

Also, an experiment was conducted to determine where the anionic LPXs localize upon administration to mice. As shown in FIG. 8, anionic LPXs localized to the spleens of animals upon administration, consistent with previous studies (Krantz et al, Nature 534: 396-401 (2016)).

RNA LPX, anionic lipoplex (LPX) or multilamellar RNA-NPs were administered to mice and spleens were harvested one week later for assessment of activated DCs (*p<0.05 unpaired t test). The RNA used in this experiment was tumor-derived mRNA from the K7M2 tumor osteosarcoma cell line. As shown in FIG. 2F, mice treated with multilamellar RNA NPs exhibited the highest levels of activated DCs.

Anionic tumor mRNA-lipoplexes, tumor mRNA-lipoplexes, and multilamellar tumor mRNA loaded NPs were compared in a therapeutic lung cancer model (K7M2) (n=5-8/group). Each vaccine was intravenously administered weekly (×3) (**p<0.01, Mann Whitney). The % CD44+CD62L+ of CD8+ splenocytes is shown in FIG. 2G and the % CD44+CD62L+ of CD4+ splenocytes is shown in FIG. 2H. Also, FIG. 2J shows that multilamellar (ML) RNA-NPs mediate substantially increased IFN-alpha which is an innate anti-viral cytokine. This demonstrates that ML RNA-NPs allow for substantially greater innate immunity which is enough to drive efficacy from even non-antigen specific ML RNA-NPs. Taken together, these figures demonstrate the superior efficacy of multilamellar tumor specific RNA-NPs, relative to anionic LPX and RNA LPX.

Anionic tumor mRNA-lipoplexes, cationic tumor mRNA-lipoplexes and multilamellar tumor mRNA loaded NPs were compared in a therapeutic lung cancer model (K7M2) (n=8/group). Each vaccine was iv administered weekly (×3), *p<0.05, Gehan Breslow-Wilcoxon test. The percent survival was measured by Kaplan-Meier Curve analysis. As shown in FIG. 2I, multilamellar tumor specific RNA-NPs mediated superior efficacy, compared to cationic RNA lipoplexes and anionic RNA lipoplexes, for increasing survival.

Herein it is demonstrated that the multilamellar RNA-NP formulation targeting physiologically relevant tumor antigens is more immunogenic (FIGS. 2F-2H, 2J) and significantly more efficacious (FIG. 2I) compared with anionic LPX and RNA LPX. Without being bound to any particular theory, by altering RNA-lipid ratios and increasing the zeta potential, a novel RNA-NP design composed of multi-lamellar rings of tightly coiled mRNA has been developed (FIG. 1C), which multi-lamellar design is thought to facilitate increased NP uptake of mRNA (condensed by alternating positive/negative charge) for enhanced particle immunogenicity and widespread in vivo localization to the periphery and tumor microenvironment (TME). Systemic administration of these multi-lamellar RNA-NPs localize to lymph nodes, reticuloendothelial organs (i.e., spleen and liver) and to the TME, activating DCs therein (based on increased expression of the activation marker CD86 on CD11c+ cells). These activated DCs prime antigen specific T cell responses, which lead to anti-tumor efficacy (with increased TILs) in several tumor models.

### Example 5

This example demonstrates the ability of multilamellar RNA-NPs to systemically activate DCs, induce antigen specific immunity and elicit anti-tumor efficacy.

The effect of multilamellar RNA NPs were tested in a second model. Here, BALB/c mice (8 mice per group) inoculated with K7M2 lung tumors were vaccinated thrice-weekly with multilamellar RNA-NPs. A control group of mice was untreated. The lungs were harvested one week after the 3rd vaccine for analysis of intratumoral memory T cells ***p<0.001, Mann Whitney test. FIG. 3A provides a pair of photographs of RNA-NP treated-lungs (left) and of untreated lungs (right). FIG. 3B is a graph of the % central memory T cells (CD62L+CD44+ of CD3+ cells) in the harvested lungs of untreated mice, mice treated multilamellar RNA NPs with GFP RNA, and mice treated multilamellar RNA NPs with tumor-specific RNA.

Also, BALB/c mice or BALB/c SCID (Fox Chase) mice (8 mice per group) were inoculated with K7M2 lung tumors and vaccinated intravenously thrice-weekly with multilamellar RNA-NPs comprising GFP RNA or tumor-specific RNA. A control group of mice was untreated. % survival was plotted on a Kaplan-Meier curve (***p<0.0001, Gehen-Breslow-Wilcox). As shown in FIG. 3C, the percent survival of BALB/c mice treated with multilamellar RNA NPs with tumor-specific RNA was highest among the three groups. Interestingly, the percent survival of BALB/c SCID (Fox Chase) mice treated with multilamellar RNA NPs with GFP RNA was about the same as mice treated with multilamellar RNA NPs with tumor-specific RNA (FIG. 3D).

Taken together, the data of FIGS. 3A-3D demonstrate that monotherapy with RNA-NPs comprising GFP RNA or tumor-specific RNA mediates significant anti-tumor efficacy against metastatic lung tumors in immunocompetent animals and SCID mice. In BALB/c mice bearing metastatic lung tumors (FIG. 3A-3D), both GFP (control) and tumor specific RNA-NPs mediate innate immunity and anti-tumor activity; however, only tumor specific RNA-NPs mediate increases in intratumoral memory T cells and long-term survivor outcome (FIG. 3A-3D). Anti-tumor activity of RNA-NPs in mice bearing intracranial malignancies was also demonstrated (data not shown).

These data demonstrate that multilamellar RNA-NPs systemically activate DCs, induce antigen specific immunity and elicit anti-tumor efficacy. FIGS. 3A-3D shows that control RNA-NPs elicit innate response with some efficacy that is not as robust as tumor specific RNA-NPs. Compared with untreated mice, no effects of uncomplexed NPs have been observed, but both non-specific (GFP RNA) and tumor-specific RNA when incorporated into multilamellar RNA NPs mediate innate immunity; however only tumor specific RNA-NPs elicit adaptive immunity that results in a long-term survival benefit (Figure. 3A-3D).

### Example 6

This example demonstrates personalized tumor RNA-NPs are active in a translational canine model.

The safety and activity of multilamellar RNA-NPs was evaluated in client-owned canines (pet dogs) diagnosed with malignant gliomas or osteosarcomas. The malignant gliomas or osteosarcomas from dogs were first biopsied for generation of personalized tumor RNA-NP vaccines.

To generate personalized multilamellar RNA NPs, total RNA materials was extracted from each patient's biopsy. A cDNA library was then prepared from the extracted total RNA, and then mRNA was amplified from the cDNA library. mRNA was then complexed with DOTAP lipid NPs, into multilamellar RNA-NPs as essentially described in Example 1. Blood was drawn at baseline, then 2 hours and 6 hours post-vaccination for assessment of PD-L1, MHCII, CD80, and CD86 on CD11c+ cells. CD11c expression of PD-L1, MHC-II, PDL1/CD80, and PD-L1/CD86 is plotted over time during the canine's initial observation period. CD3+ cells were analyzed over time during the canine's initial observation period for percent CD4 and CD8, and these subsets were assessed for expression of activation markers (i.e., CD44). From these data, it was shown that multilamellar RNA-NPs elicited an increase in 1) CD80 and MHCII on CD11c⁺ peripheral blood cells demonstrating activation of peripheral DCs; and 2) an increase in activated T cells.

Interestingly, within a few hours after administration, tumor specific RNA-NPs elicited margination of peripheral blood mononuclear cells, which increased in the subsequent days and weeks post-treatment; suggesting that RNA-NPs mediate lymphoid honing of immune cell populations before egress.

These data demonstrated that personalized mRNA-NPs are safe and active in translational canine disease models.

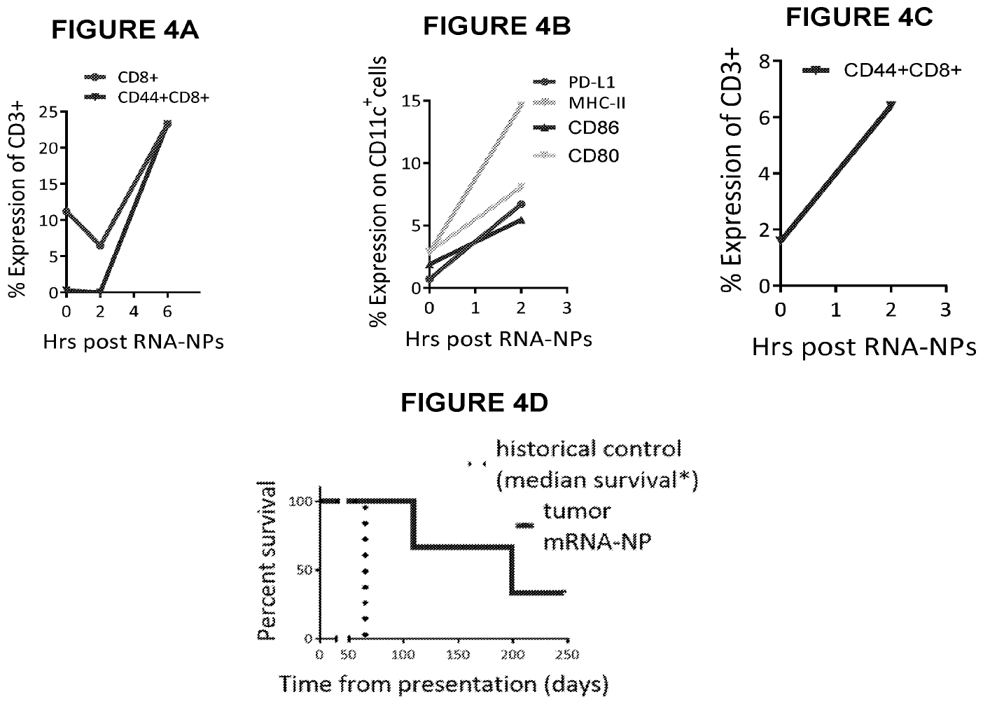

Specific data from canines evaluated in this manner are shown. A 31 kg male Irish Setter was enrolled on study per owner's consent to receive multilamellar RNA-NPs. Tumor mRNA was successfully extracted and amplified after tumor biopsy. Immunologic response was plotted in response to 1^{st} vaccine. The data show increased activation markers over time on CD11c+ cells (DCs) (FIG. 4A), The data show increased CD8+ cells that are activated (CD44+CD8+ cells) within the first few hours post RNA-NP vaccine. These data support that the multilamellar RNA-NPs are immunologically active in a male Irish Setter. A male boxer diagnosed with a malignant glioma was enrolled on study per owner's consent to receive RNA-NPs. Tumor mRNA was successfully extracted and amplified after tumor biopsy. Immunologic response is plotted in response to 1^{st} vaccine (FIG. 4B). The data show increased activation markers over time on CD11c+ cells (DCs). As shown in FIG. 4C, an increase in activated T cells (CD44+CD8+ cells) was observed within the first few hours post RNA-NP vaccine. These data support that the multilamellar RNA-NPs are immunologically active in a male canine boxer.

After receiving weekly RNA-NPs (×3), the canines diagnosed with malignant gliomas had a steady course. Post vaccination MRI showed stable tumor burdens, with increased swelling and enhancement (in some cases), which may be more consistent with pseudoprogression from an immunotherapeutic response in otherwise asymptomatic canines. Survival of canines diagnosed with malignant gliomas receiving only supportive care and tumor specific RNA-NPs (following tumor biopsy without resection) is shown in FIG. 4D. In FIG. 4D, the median survival (shown as dotted line) was about 65 days and was reported from a meta-analysis of canine brain tumor patients receiving only symptomatic management. In a previous study, cerebral astrocytomas in canines has been reported to have a median overall survival of 77 days. The personalized, multilamellar RNA NPs allowed for survival past 200 days.

Aside from low-grade fevers that spiked 6 hrs post-vaccination on the initial day, personalized tumor RNA-NPs (1×) were well tolerated with stable blood counts, differentials, renal and liver function tests. To date, we have treated four client-owned canines diagnosed with malignant brain tumors. It is important to highlight that these canines received no other therapeutic interventions for their malignancies (i.e., surgery, radiation or chemotherapy), and all patients assessed developed immunologic response with pseudoprogression or stable/smaller tumors. One canine was autopsied after RNA-NP vaccines. In this patient there were no toxicities believed to be related to the interventional agent.

These results suggest safety and activity of tumor specific RNA-NPs in client-owned canines with malignant brain tumors for subjects that did not receive any other anti-tumor therapeutic interventions.

### Example 7

This example demonstrates toxicology study of murine glioma mRNA and pp65 mRNA encapsulated in DOTAP liposomes after intravenous delivery to C57BL/6 mice.

The objective of this study was to evaluate the safety of pp65 mRNA encapsulated by DOTAP liposomes when delivered intravenously in C57BL/6 mice. Experimental procedures applicable to pathology investigations are summarized in Table 1. All interim phase animals were submitted for necropsy on Day 35±1 day. Necropsies were performed by University of Florida personnel. Tissue samples listed in Table 2 were collected and fixed in 10% neutral buffered formalin, unless otherwise noted; tissues from the early death animal were fixed in 10% neutral buffered formalin.

TABLE 1

| | | Total Dose | Number of Mice | | | | | |
|---|---|---|---|---|---|---|---|---|
| | | (total mRNA + LP) | Day 35 ± 1 day | | Day 56 ± 2 | | Day 112 ± 3 days | |
| Group | Treatment a | (mg/kg) | Males | Female | Males | Female | Males | Female |
| 1 | Vehicle | 0 | 5 | 5 | 5 | 5 | 5 | 5 |
| 2 | LP | 0 + 15.0 | 5 | 5 | 5 | 5 | 5 | 5 |
| 3 | RNA + LP | 0.2 + 3.0 | 5 | 5 | 5 | 5 | 5 | 5 |
| 4 | RNA + LP | 1.0 + 15.0 | 5 | 5 | 5 | 5 | 5 | 5 |

TABLE 2
Tissue Collection and Examination

| Provantis Tissue Term | Protocol Tissue Term | Collect | Microscopic | Comment |
|---|---|---|---|---|
| BONE, FEMUR | Femur with bone | X | X | |
| | marrow (R) | | | |
| BONE MARROW | | X | X | |
| BONE, STERNUM | Sternum | X | X | |
| BRAIN | Brain stem | X | X | |
| | Cerebellum | | | |
| | Cerebrum | | | |
| EPIDIDYMIS | Epididymis | X | X | |
| ESOPHAGUS | Esophagus | X | X | |
| EYE | Eye with optic | X | X | Fixed in modified |
| | nerve (R) | | | Davidson's solution |
| NERVE, OPTIC | | X | X | |
| GLAND, ADRENAL | Adrenal gland (R) | X | X | |
| GLAND, | | X | X | |
| PARATHYROID | | | | |
| GLAND, THYROID | Thyroid/parathyroid | X | X | |
| GLAND, PITUITARY | Pituitary | X | X | |
| GLAND, PROSTATE | Prostate | X | X | |
| GLAND, SALIVARY | Salivary gland (R, | X | X | |
| | mandibular) | | | |
| GLAND, SEMINAL | Seminal vesicles | X | X | |
| VESICLE | | | | |
| HEART | Heart | X | X | |
| KIDNEY | Kidney (R) | X | X | |
| LARGE INTESTINE, | Cecum | X | X | |
| CECUM | | | | |
| LARGE INTESTINE, | Colon | X | X | |
| COLON | | | | |
| LARGE INTESTINE, | Rectum | X | X | |
| RECTUM | | | | |
| LIVER | Liver | X | X | |
| LUNG | Lungs | X | X | |
| LYMPH NODE, | Lymph node | X | X | |
| MESENTERIC | (mesenteric) | | | |
| MUSCLE, DIAPHRAGM | Diaphragm | X | X | |
| MUSCLE, | Quadriceps (R) | X | X | |
| QUADRICEPS | | | | |
| NERVE, SCIATIC | Sciatic nerve (R) | X | X | |
| OVARY | Gonad (Ovary, R) | X | X | |
| PANCREAS | Pancreas | X | X | |
| SITE, INJECTION | Tail (injection site) | X | X | |
| SKIN | Skin | X | X | |
| SMALL INTESTINE, | Duodenum | X | X | |
| DUODENUM | | | | |
| SMALL INTESTINE, | Ileum | X | X | |
| ILEUM | | | | |
| SMALL INTESTINE, | Jejunum | X | X | |
| JEJUNUM | | | | |
| SPINAL CORD | Spinal cord, cervical | X | X | |
| | Spinal cord, lumbar | | | |
| | Spinal cord, thoracic | | | |
| SPLEEN | Spleen | X | X | |
| STOMACH | Stomach | X | X | |
| TESTIS | Gonad (Testis, R) | X | X | Fixed in modified |
| | | | | Davidson's solution |
| THYMUS | Thymus | X | X | |
| TONGUE | Tongue | X | X | |
| URINARY BLADDER | Urinary bladder | X | X | |
| UTERUS | Uterus | X | X | |
| VAGINA | Vagina | X | X | |
| — | Gross lesions | X | X | |

Tissues required for microscopic evaluation were trimmed, processed routinely, embedded in paraffin, and stained with hematoxylin and eosin by Charles River Laboratories Inc., Skokie, Ill. Light microscopic evaluation was conducted by the Contributing Scientist, a board-certified veterinary pathologist on all protocol-specified tissues from all animals in Groups 1 and 4, and any early death animals.

Tissues that were supposed to be microscopically evaluated per protocol but were not available on the slide (and therefore not evaluated) are listed in the Individual Animal Data of the pathology report as not present. These missing tissues did not affect the outcome or interpretation of the pathology portion of the study because the number of tissues examined from each treatment group was sufficient for interpretation.

Gross Pathology: No test article-related gross findings were noted. The gross findings observed were considered incidental, of the nature commonly observed in this strain and age of mouse, and/or were of similar incidence in control and treated animals and, therefore, were considered unrelated to administration of a 1:1 ratio of pp65 mRNA and KR158mRNA in DOTAP liposomes.

Histopathology: No test article-related microscopic findings were noted. There were a few animals with inflammatory cell infiltrates at the injection site; this finding is common for injection sites and at this point in the study, was considered equivocal. The microscopic findings observed were considered incidental, of the nature commonly observed in this strain and age of mouse, and/or were of similar incidence and severity in control and treated animals and, therefore, were considered unrelated to administration of a 1:1 ratio of pp65 mRNA and KR158mRNA in DOTAP liposomes.

It was concluded that intravenous injection into the tail vein of mice of 1.0 mg/kg KR158 and pp65 mRNAs+15.0 mg/kg DOTAP liposome on Study Days 0, 14, and 28 resulted in no gross or microscopic test article-related findings on Study Day 35±1 day. There were small amounts of inflammatory cell infiltrates at the injection site, which is a common finding for injection sites. This finding was equivocal.

### Example 8

This example describes a study aimed at determining the impact of pDCs transfected with multilamellar RNA-NPs on antigen specific T-cell priming.

While pDCs are well-known stimulators of innate immunity and type I IFN, they also mediate profound effects on intratumoral adaptive immunity. They can: 1) directly present antigen for priming of tumor specific T cells; 2) assist adaptive response through chemokine recruitment of other DC subtypes (via chemokines CCL3, CCL4, CXCL10); 3) polarize Th1 immunity through IL-12 secretion; and/or 4) mediate tumor antigen shedding (through cytokine, TRAIL or granzyme B) for DC loading and T cell priming. Despite these effector functions, pDCs may also dampen immunity through release of immunoregulatory molecules (IL-10, TGF-β, and IDO) and promotion of regulatory T cells (Tregs). The purpose of this study is to elucidate the effects of RNA-NP transfected-pDCs on adaptive immunity and antigen specific T cell priming. It is hypothesized that RNA-NP activated pDCs serve as direct primers of antigen specific immunity and assist classical DCs (cDCs) and/or myeloid-derived DCs (mDCs) in promoting effector T-cell response. These experiments are to shed new light on the activation state of pDCs requisite for RNA-NP mediated immunity and their exhaustion over time that may be co-opted for enhanced immunotherapeutic effect.

Statistical Analyses

In the study of Example 9.1 where survival is of interest, the log-rank test is used to compare Kaplan-Meier survival curves between treatment groups and control groups. Experience with our tumor models indicates that median overall survival in untreated control mice is approximately 30 days, with survival times following a Weibull distribution with shape parameter k=6. As an example, with 10 mice each in 2 tumor-bearing groups (treated and untreated), comparison of survival curves using a one-sided log-rank test evaluated at 0.05 significance has at least 80% power to detect an improvement in median survival of 8 days in the treated group compared to the untreated group. This effect size was determined by simulating 1000 Weibull-distributed survival datasets with shape parameter k=6 under the alternative hypothesis effect size and then observed the proportion of log-rank tests of these datasets that were significant at p<0.05. In the studies of Examples 9.2-9.4, responses observed at different times are analyzed using a two-way ANOVA model with mutually exclusive groups distributed among treatments and observation times. Change in immune response parameters over time are assessed using generalized linear mixed effect models (GLMMs). Response variables for experiments that are completely replicated at least once are analyzed using GLMMs. Experimental replication are modeled as a random effect to account for “batch” or “laboratory day” variability. Treatment and control groups are modeled as fixed effects and compared using ANOVA-type designs nested within the mixed effect modeling framework.

### Example 8.1

This example describes an experiment designed to determine anti-tumor efficacy of RNA-NPs in wild-type and pDC KO mice.

Tumorgenicities for KR158b-luc, GL261-luc and a murine H3.3K27M mutant cell line have been set up. KR158b-luc and GL261-luc are both transfected with luciferase so that tumors can be monitored for growth using bioluminescent imaging. Tumorigenic dose of KR158b-luc and the H3K27M mutant line is 1×10⁴ cells. Tumorigenic dose of GL261-luc is 1×10⁵ cells. GL261 and KR158 are injected into the cerebral cortex of C57Bl/6 (3 mm deep into the brain at a site 2 mm to the right of the bregma); H3K27M glioma cells are injected midline. Tumor mRNA is extracted from the parental cell lines (i.e., KR158b without luciferase) for vaccine formulation consisting of an intravenous (iv) injection of 25 μg of tumor specific mRNA complexed with 375 μg of our custom lipid-NP formulation (per mouse). These are compared simultaneously to 10 negative control mice receiving NPs alone and nonspecific (i.e., pp65 mRNA) RNA-NPs. Mice are vaccinated 3 times at 7-day intervals beginning 5 days after tumor implantation. IFN-α levels are assessed from serum of wild-type and pDC KO mice at serial time points (5 d, 12 d, and 19 d). In wild-type mice who develop treatment response, but succumb to disease, the immunologic escape mechanisms in tumors (i.e., expression of checkpoint ligands, IDO, downregulation of MHC class I) and within the tumor microenvironment (i.e., MDSCs, Tregs, and TAMs) are explored.

Based on preclinical data demonstrating anti-tumor activity of RNA-NPs in these models, it is anticipated that anti-tumor activity is abrogated in pDC KO mice.

### Example 8.2

This example describes an experiment designed to determine the pDC phenotype and function following activation by RNA-NPs.

To assess pDC phenotype, KR158b bearing C57BI/6 mice are vaccinated with TTRNA-NPs composed from 375 μg of FITC labeled DOTAP (Avanti) with 25 μg of TTRNA (derived from KR158b and delivered iv). Twenty-four hours after vaccination recipient mice are euthanized (humanely killed with C02) for collection of spleens, tumor draining lymph nodes (tdLNs) and tumors. Organs are digested into a single cell suspension, undergo RBC lysis (PharmLyse, BD Bioscience) before incubation at 37° C. for 5 minutes. Ficoll gradients are used to separate WBCs from parenchymal cells. The cells at the interface are collected, washed, and analyzed. pDCs are stained for CD11c, B220 and Gr-1 (ebioscience). Distinct pDC subsets are identified by differential staining for CCR9, SCA1, and Ly49q. Activation state is assessed based on expression of co-stimulatory molecules (i.e. CD40, CD80, CD86) chemokines (i.e. CCL3, CCL4, CXCL10) and chemokine receptors (i.e. CCR2, CCR5, CCR7). Detection secondary antibody is rabbit IgG conjugated with AlexaFlour®488 (ThermoFisher Scientific) for FITC detection. Effector versus regulatory function is determined through intracellular staining for effector (i.e. IFN-I, IL-12) versus regulatory cytokines (i.e. TGF-β, IL-10). Analyses will be conducted by multi-parameter flow cytometry (LSR, BD Bioscience) and immunohistochemistry (IHC).

Based on our preliminary data showing substantial increases in pDCs in peripheral and intratumoral organs, it is expected to identify FITC positive pDCs in the spleen, tdLNs and intracranial tumors.

### Example 8.3

This example describes an experiment designed to determine whether RNA-NP transfected pDCs mediate direct or indirect activation of antigen specific T cells.

While pDCs are well known stimulators of innate immunity and type I IFN, their cumulative effects on antigen specific responses are still being uncovered. Since they express MHC class II, they have APC capacity, but compared to their cDC counterparts, they are believed to be poor direct primers of antigen specific immunity. This experiment is aimed at yielding a better understanding of pDCs, in the context of RNA-NPs, as either direct primers or facilitators of antigens specific immunity. To determine the effects of pDCs on antigen specific T cells, KR158b bearing mice are vaccinated with TTRNA (derived from the murine glioma line KR158b) encapsulated into FITC-labeled NPs (Avanti), and FACSort (BD Aria II) relevant FITC+ pDCs from spleens, tdLNs and intracranial tumors (as indicated above). RNA-NP transfected pDCs are then co-cultured with naïve magnetically separated CD4 and CD8 T cells, and T cells are assessed for proliferation, phenotype (effector vs central memory), function and cytotoxicity. Indirect effects from pDCs are assessed via ex vivo co-cultures with TTRNA-loaded DCs (matured ex vivo from murine bone marrow) with naïve CD4 and CD8 T cells. Ex vivo co-cultures will be performed in triplicate, for 7 days in a 96 well plate with naïve T cells (40,000 RNA-NP transfected pDCs with 400,000 T cells) labeled with CFSE (Celltrace, Life Technologies). T cell proliferation is determined by measuring CFSE dilution by flow cytometry. Phenotype for effector and central memory populations is determined through differential staining for CD44 and CD62L. These T cells are re-stimulated for a total of 2 cycles before supernatants are harvested for detection of Th1 cytokines (i.e. IL-2, TNF-α, and IFN-γ) by bead array (BD Biosciences). Stimulated T cells are also incubated in the presence of KR158b (stably transfected with GFP) or control tumor (B16F10-GFP) and assessed for their ability to induce cytotoxicity. Amount of GFP in each co-culture, as a surrogate for living tumor cells, are quantitatively measured by flow cytometry.

The in vivo effects of FACSorted RNA-NP transfected pDCs are determined by adoptively transferring these cells (250,000 cells/mouse) to tumor-bearing mice (weekly ×3) and harvesting spleens, tdLNs, and tumors one week later for assessment of antigen specific T cells by YFP expression in IFN-γ reporter mice (GREAT mice, B6 transgenic, containing IFN-γ promotor with IRES-eYFP reporter, Jackson labs). In separate experiments, IFN-γ reporter mice are vaccinated with TTRNA-NPs with and without pDC depleting mAbs before harvesting spleens, tdLNs, and intracranial tumors one week later for determination of antigen specific T cells by YFP expression. T cell functional assays are performed as described above.

It is anticipated that these pDCs are requisite for priming antigen specific T cells through either direct and/or indirect means.

### Example 8.4

This example describes an experiment designed to determine whether RNA-NP activated pDCs promote antigen specific T cell priming from cDCs and/or mDCs.

While IFN-I release from pDCs is known to increase activation markers on cDCs and mDCs, the role of pDCs on direct T cell priming from cDCs/mDCs is less clear. This experiment is aimed at elucidating the ability of RNA transfected cDCs and mDCs to prime antigen specific T cells in the presence or absence of activated pDCs. To determine effects of pDCs on other DC subsets, KR158b bearing C57Bl/6 and pDC knock out (KO) mice (BDCA2-DTR, B6 transgenic mice, Jackson labs) are vaccinated and T cell priming from cDCs and mDCs are assessed. FITC+cDC and mDC populations are sorted via FACSort within 24 h of iv TTRNA-NPs (FITC-labeled) and are evaluated for their ability to prime naïve T cell responses in vitro based on proliferation, functional and cytotoxicity assays. Resident and migratory cDCs are identified by CD11c+CD103+MHCII+ cells and CD11c+CD11b+MHCII+ cells respectively; mDCs are identified by CD11c+CD14+MHCII+ cells. Cytokines, chemokines and activation markers are analyzed as described in Example 9.1. In vivo effects of these cDC/mDC are carried out in cell transfer experiments as described in Example 9.2. Briefly, FACSorted cDCs and mDCs from TTRNA-NP vaccinated C57Bl/6 mice or pDC KO mice are adoptively transferred (250,000 cells/mouse) to tumor-bearing mice (once weekly ×3) before harvesting spleens, tdLNs, and intracranial tumors one week later for assessment of antigen specific T cells by YFP expression in IFN-γ reporter mice. Proliferation, functional and cytotoxicity assays are performed.

It is expected that ML RNA-NPs activate pDCs which enhance activation phenotype and direct priming of T cells from cDCs and mDCs.

If a lack of indirect effects from pDCs on cDCs and/or mDCs, pDC® effects on NK cells are evaluated including their activation state, function, and cytotoxicity.

### Example 8.5

This example describes an experiment designed to determine how pDCs influence effector/regulatory T cells over time within the intratumoral microenvironment.

Recruitment of pDCs to tumors is typically associated with a regulatory phenotype characterized by increased IDO, FoxP3+Tregs and secretion of immunoregulatory cytokines. In this experiment, it is determined whether RNA-NP activated pDCs function distinctly by activating T cells over time in the tumor microenvironment. To determine intratumoral effects of pDCs, TTRNA-NPs are administered to KR158b bearing IFN-γ reporter mice with and without pDC depleting mAbs (Bioxcell). Activated and regulatory T cells are assessed over time in the intratumoral microenvironment at serial time points (6 h, 1 d, 7 d, and 21 d). Effector T cells are characterized, and Tregs are phenotyped through expression of FoxP3, CD25, and CD4. pDCs from non-depleted animals will be FACSorted from these sites and are phenotyped for expression of cytokines, chemokines, activation markers (i.e., CD80, CD86, CD40), cytolytic markers (i.e. TRAIL, granzyme b) and regulatory markers (i.e., IL-10, TGF-β, IDO). Immunophenotypic changes by tumor cells are also assessed over time (i.e., MHC-I, PD-L1, SIRPα).

### Example 9

This example describes a study aimed at evaluating the role of type I interferons on RNA-NP activated T-cell egress, trafficking and function.

Statistical Analysis Tumor-bearing mice are randomized prior to receiving interventional treatments. The choice of 10 animals per group should yield adequate power for detecting effects of interest. As an example, within an ANOVA design with 7 treatment groups observed at a particular time, a pairwise contrast performed within the ANOVA framework can detect an effect size equal to 1.27 SD units with 80% power at a 2-sided significance level of 0.05. Immune parameter responses observed in experimental groups at several observation times are analyzed using generalized linear models (GLMs) with normal or negative binomial response errors. Responses are organized in a two-way ANOVA design with mutually exclusive groups distributed among treatments and observation times. Response variables for experiments that are completely replicated at least once are analyzed using GLMMs. Experimental replication are modeled as a random effect to account for “batch” or “laboratory day” variability. Treatment and control groups are modeled as fixed effects and compared using ANOVA-type designs nested within the mixed effect modeling framework.

### Example 9.1

This example describes an experiment designed to determine the chemokine receptor, S1P1, and VLA-4/LFA-1 expression profile of antigen specific T cells after RNA-NP vaccination.

IFN-I's effects on sphingosine-1-phosphate receptor 1 (S1P1), which is necessary for T cell egress from lymphoid organs, and integrins (i.e. VLA-4, LFA-1) necessary for T cell traversion across the BBB are assessed. KR158b bearing IFN-γ reporter mice, or IFN-γ reporter mice receiving IFNAR1 blocking mAbs (Bioxcell) are implanted with TTRNA-NPs. RNA-NPs composed from 375 μg of DOTAP (Avanti) with 25 μg of TTRNA (extracted from KR158b and delivered iv) are administered once weekly (×3) and are begun 5 days after implantation. One week after the last vaccine, recipient mice are euthanized (humanely killed with CO₂) and spleens, tdLNs, bone marrow, and intracranial tumors are harvested. Organs are digested, and antigen specific T cells from spleens, lymph nodes, bone marrow and tumors are identified by YFP expression and by differential staining for effector and central memory T cells (i.e., of CD62L and CD44) at serial time points (7, 14 and 21 days). Th1-associated chemokine receptors (i.e., CCR2, CCR5, CCR7 and CXCR3), S1P1 expression, VLA-4, and LFA-1 expression (ebioscience) from CD4 and CD8 T cells are assessed by multi-para meter flow cytometry and IHC.

It is expected that LFA-1 and CCR2 are expressed on activated T cells following RNA-NP administration. If no changes in chemokine expression pattern, S1P1 and integrins on activated T cells after IFNAR1 mAbs, RNA-seq analysis is performed on FACS sorted T cells (YFP+ cells) from mice treated with and without IFNAR1 mAbs and assess changes in immune related genes.

### Example 9.2

This example describes an experiment designed to determine the effects of IFN-I on in vitro and in vivo migration of RNA-NP activated T cells.

Based on our data demonstrating increased antigen specific T cells in peripheral organs but lack of anti-tumor efficacy after IFNAR1 blockade, IFN-I's effects on RNA-NP activated T cell migration are determined. KR158b bearing IFN-γ reporter mice, or IFN-γ reporter mice receiving IFNAR1, LFA-1 or CCR2 blocking antibodies are vaccinated with iv TTRNA-NPs once weekly (×3). In vivo traversion across the BBB is assessed from percentage and absolute numbers of T cells in intracranial tumors (relative to spleen, lymph nodes and bone marrow) at serial time points (5 d, 10 d, 15 d, 20 d post RNA-NPs).

The migratory capacity of T cells are also analyzed via in vitro cultures. KR158b tumor bearing naïve, INFAR1, LFA-1 or CCR2 KO animals (B6 transgenic, Jackson) are vaccinated with iv TTRNA-NPs. T cells are FACSorted via a BD Aria II Cell Sorter into a 50-100% FBS solution. These T cells are assessed for migratory capacity in transwell assays (ThermoFisher Scientific). Briefly, T cells are placed in the upper layer of a cell culture insert with a permeable membrane in between a layer of KR158b-GFP tumor cells. Migration is assessed by number of cells that shift between layers. T cells are plated in T cell media with and without IL-2 (1 microgram/mL) at a concentration of 4×10⁶ per mL for co-culture with tumor cells (4×10⁶/mL) (×48 hrs) before determination of IFN-γ by ELISA (ebioscience). Amount of GFP in each co-culture, as a surrogate for living tumor cells, is quantitatively measured by flow cytometric analysis.

It is anticipated that type I IFNs are necessary for activated T cell trafficking across the BBB. If there is an inability to adequately define antigen specific T cells, the response against a physiologically relevant GBM antigen, pp65, which will be spiked into our tumor mRNA cohort, is tracked in HLA-A2 transgenic mice by overlapping peptide pool re-stimulation assays and through analysis for pp65-HLA-A2 restricted epitope NTUDGDDNNDV by tetramer staining for CD8+ cells in spleens, tdLNs and intracranial tumors.

### Example 9.3

This example describes an experiment designed to delineate the contribution of IFN-I on antigen specific T cell function following RNA-NPs.

IFN-Is have been shown to promote Tregs and regulate effector and memory CD8+ cells, but they are also essential in promoting activated T cell responses following RNA-NP vaccination. Due to these distinct effects, the contribution of IFN-I on antigen specific T cell function following RNA-NP vaccines is determined. KR158b bearing IFN-γ reporter mice, or IFN-γ reporter mice receiving IFNAR1 mAbs, are vaccinated with iv TTRNA-NPs once weekly (×3). Antigen specific T cells are assessed by YFP+ cells. YFP+ T cells from spleens, lymph nodes, bone marrow and tumor are assessed for their activation status (i.e. CD107a, perforin, granzyme), proliferation (through fluorescent dilution of adoptively transferred cells labeled with CellTrace Violet), differentiation (into effector and central memory subsets, and cytotoxicity. T cell cytotoxicity is determined in the presence of KR158b (stably transfected with GFP) or control tumor (B16F10). It is also expected that type I IFNs enhance T cell proliferation and function within the tumor microenvironment.

If no changes in migratory capacity or function of antigen specific T cells after blockade of type I IFN, the effects of type I IFN on modulating T cell exhaustion is assessed. the effects of type I IFNs on expression of immune checkpoints (i.e. PD-1, TIM-3, LAG-3) and their ligands on tumor cells and APCs (i.e. PD-L1, galectin-9) is also evaluated.

### Example 10

This example demonstrates non-antigen specific multilamellar (ML) RNA NPs mediate antigen-specific immunity long enough to confer memory and fend off re-challenge of tumor.

An experiment was carried out with long-term surviving mice (e.g., mice that survived for ˜100 days) that were challenged a total of two times via tumor inoculation, but treated only once weekly (×3) with ML RNA NPs comprising GFP RNA or pp65 RNA (each of which were non-specific to the tumor) or with ML RNA NPs comprising tumor-specific RNA. The treatment occurred just after the first tumor inoculation and about 100 days before the second tumor inoculation. Because none of the control mice (untreated mice) survived to 100 days, a new control group of mice were created by inoculating the same type of mice with K7M2 tumors. The new control group like the original control mice did not receive any treatment. The long-time survivors also did not receive any treatment after the second time of tumor inoculation. A timeline of the events of this experiment are depicted in FIG. 7A.

Remarkably, mice in all 3 groups contained long-time survivors that survived the second tumor challenge. As shown in FIG. 7B (which shows only the time period following the 2^{nd} inoculation), mice in all 3 groups contained long-time survivors with survival to 40 days post tumor implantation (second instance of tumor inoculation). Interestingly, the percentage of long-time survivor mice that were previously treated with ML RNA NPs comprising non-specific RNA (GFP RNA or pp65 RNA) survived to 40 days post second tumor inoculation, comparable to the group treated with ML RNA NPs comprising tumor specific RNA (treated before second tumor challenge).

These data support that ML RNA NPs comprising RNA non-specific to a tumor in a subject provides therapeutic treatment for the tumor comparable to that provided by ML RNA NPs comprising RNA specific to the tumor, leading to increased percentage in animal survival.

### Example 11

This example demonstrates an exemplary method of making DOTAP coated iron oxide particles.

DOTAP-coated iron oxide particles (IONPs) were synthesized for incorporation into multilamellar RNA NPs. Briefly, a stock solution of DOTAP (about 2 to about 4 mg/ml) was prepared by dissolving DOTAP in ethanol. The DOTAP stock solution was probe-sonicated on the Q Sonica (Model: Q500), using amplitude of 38% for a total sonication time of 30 sec. An appropriate amount of DOTAP was slowly phased out to an aqueous phase, by first dissolving equal volume of sonicated DOTAP stock with equal amount of water. The resulting solution is further dissolved in water to make the final volume 10 ml. Hereinafter, this solution comprising water and DOTAP was referred to as an “aqueous DOTAP solution.”

IONPs were synthesized by thermal decomposition and coated with oleic acid which were magnetically separated to remove any free oleic acid. IONPs were finally suspended in chloroform.

An appropriate concentration of 1 ml IONP in chloroform was added to the 10 ml of the aqueous DOTAP solution. The DOTAP:Iron oxide particles had an expected ratio of 0.1:0.5, wherein 0.1 mg of DOTAP was required for coating 0.5 mg of iron oxide particles (IONP). This solution was probe sonicated at 38% amplitude, pulse in 59 s, pulse out for 10 s, strength 2000 J (Q Sonica Model: Q500). The solution was left in a fume hood under overnight constant stirring to evaporate off the organic solvent.

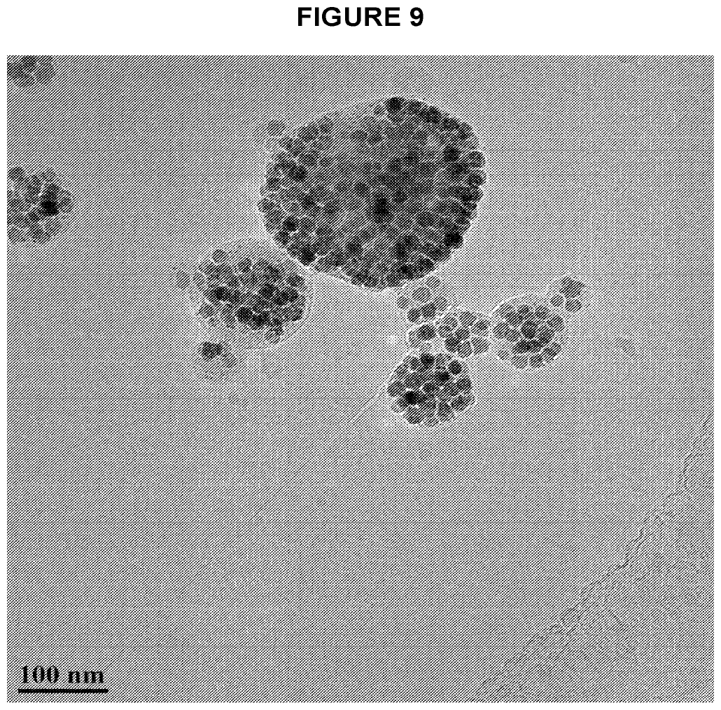

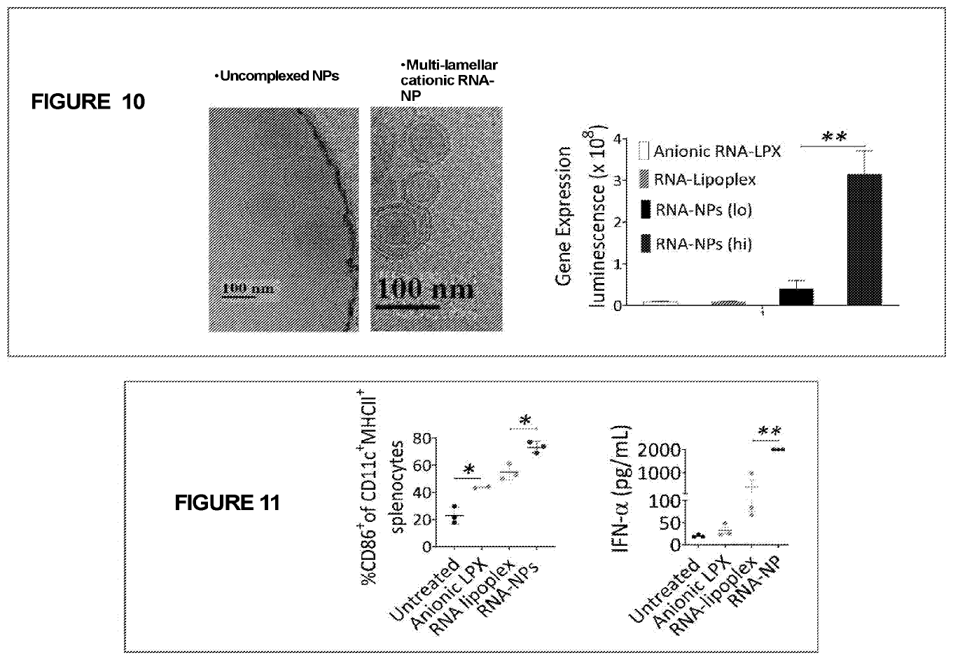

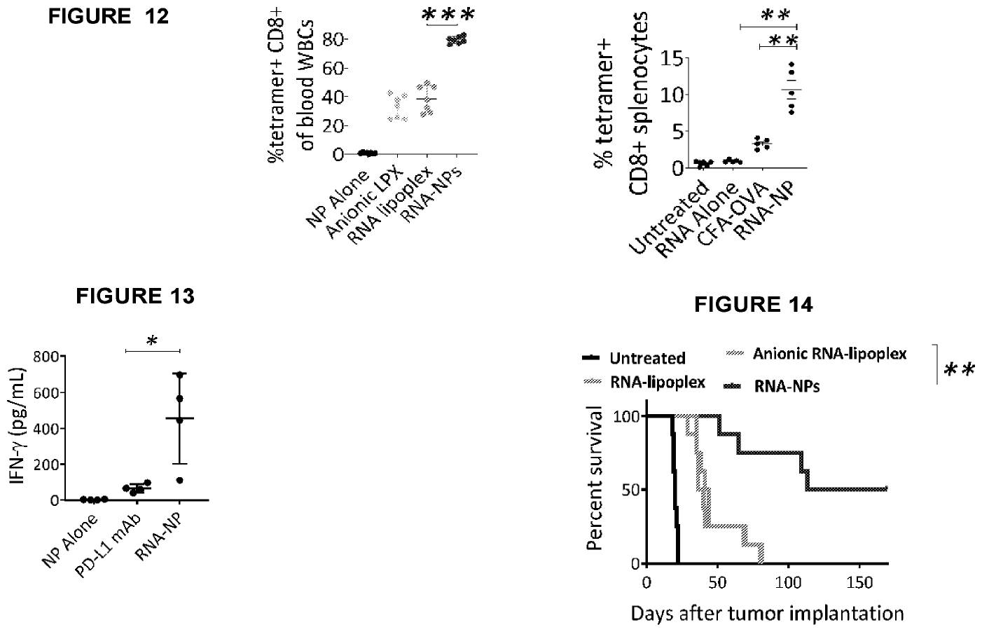

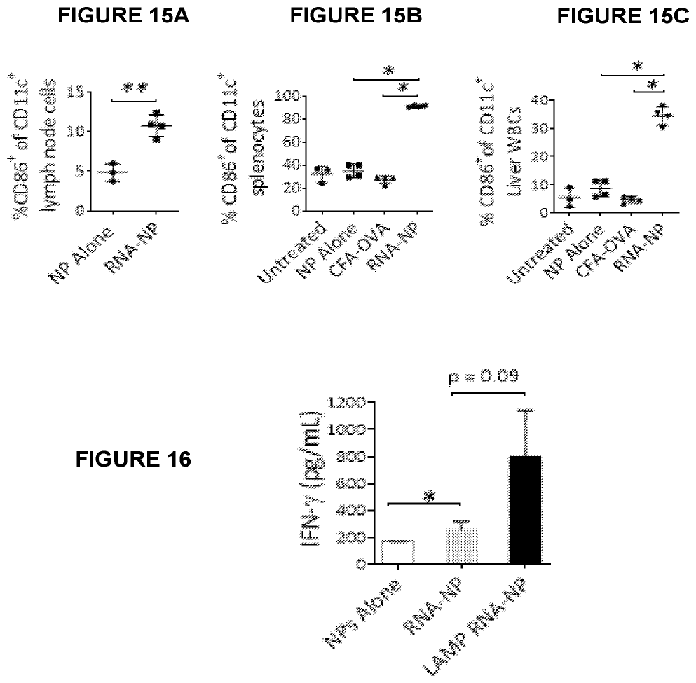

This method produced iron oxide nanoparticles held together by a lipid coating of DOTAP. The resulting particles were analyzed via transmission electron microscopy (TEM). FIG. 9 is an image of the IONPs held together by the DOTAP coating.

### Example 12

This example demonstrates a method of making multilamellar RNA NPs loaded with iron oxide nanoparticles.

Example 11 describes a method of producing oleic acid-coated IONPs held together by a coating of DOTAP, which provides the core of the multilamellar RNA NPs. The IONP core is layered with negative charge before encapsulation into multi-lamellar structures using free DOTAP without iron. Briefly, rotary vacuum evaporation is used to remove organic solvents from DOTAP/Chloroform mixtures before resuspension in aqueous solution for rotational heating, bath sonication, extrusion and layering with tumor mRNA in specific mass ratios of 1:15 (μg dosing, RNA to NP). Multi-lamellar charge is preserved by carrying out procedures in vacuum seal to prevent oxidation from ambient environment. The multilamellar RNA NPs loaded with iron oxide nanoparticles are characterized by CEM, in terms of zeta potential and RNA incorporation, as described above. Complexes are verified by Nanosight measurements of size and concentration and layers visualized by cryo-electron microscopy (CEM). Transfection in vitro is demonstrated using GFP mRNA multi-lamellar particles and immunogenicity in vivo is carried out with OVA mRNA. The transfection efficiency of multilamellar RNA NPs loaded with iron oxide nanoparticles is determined. Multilamellar RNA-NPs comprising GFP RNA loaded with and without iron oxide are used to transfect dendritic cells and the GFP positive cells are measured by flow cytometry. Bright field images and fluorescent imaging of the transfected DCs are taken.

### Example 13

This example demonstrates the effect of a magnetic field on the multilamellar RNA NPs loaded with iron oxide nanoparticles.

The effect of a 101 mT magnetic field on the ability of magnetic liposomes to deliver RNA to cells is tested. IONP-loaded multilamellar RNA NPs comprising GFP RNA are made as essentially described in Example 12. The IONP-loaded multilamellar RNA NPs are incubated with DC2.4 dendritic cells for 30 minutes in the presence or absence of a magnetic field. For one set of cells, the RNA-loaded magnetic liposomes are incubated with DC2.4 dendritic cells overnight in the absence of a magnetic field produced by a MagneFect-Nano II 24 well magnet array. After 30 minutes, particle-containing media is removed and replaced with fresh media. Gene delivery is assessed as GFP expression by flow cytometry at 24 hours. It is expected that the number of GFP+ DCs is higher when a magnetic field is present, relative to when a magnetic field is absent.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A nanoparticle comprising a positively-charged surface and an interior comprising (i) a core and (ii) at least two nucleic acid layers, wherein each nucleic acid layer is positioned between a cationic lipid bilayer.

2. The nanoparticle of claim 1, comprising at least three nucleic acid layers, each of which is positioned between a cationic lipid bilayer.

3. The nanoparticle of claim 2, comprising at least four nucleic acid layers, each of which is positioned between a cationic lipid bilayer.

4. The nanoparticle of claim 3, comprising five or more nucleic acid layers, each of which is positioned between a cationic lipid bilayer.

5. The nanoparticle of any one of claims 1 to 4, wherein the outermost layer of the nanoparticle comprises a cationic lipid bilayer.

6. The nanoparticle of any one of claims 1 to 5, wherein the surface comprises a plurality of hydrophilic moieties of the cationic lipid of the cationic lipid bilayer.

7. The nanoparticle of any one of claims 1 to 6, wherein the core comprises a cationic lipid bilayer.

8. The nanoparticle of any one of claims 1 to 7, wherein the core comprises less than about 0.5 wt % nucleic acid.

9. The nanoparticle of any one of claims 1 to 8, wherein the diameter of the nanoparticle is about 50 nm to about 250 nm in diameter, optionally, about 70 nm to about 200 nm in diameter.

10. The nanoparticle of any one of claims 1 to 9, comprising a zeta potential of about 40 mV to about 60 mV, optionally, about 45 mV to about 55 mV.

11. The nanoparticle of claim 10, comprising a zeta potential of about 50 mV.

12. The nanoparticle of any one of the preceding claims, comprising nucleic acid molecules and cationic lipid at a ratio of about 1 to about 5 to about 1 to about 20, optionally, about 1 to about 15 or about 1 to about 7.5.

13. The nanoparticle of any one of the preceding claims, wherein the cationic lipid is DOTAP or DOTMA.

14. The nanoparticle of any one of the previous claims, wherein the nucleic acid molecules are RNA molecules.

15. The nanoparticle of claim 14, wherein the RNA molecules are mRNA.

16. The nanoparticle of claim 15, wherein the mRNA is in vitro transcribed mRNA wherein the in vitro transcription template is cDNA made from RNA extracted from a tumor cell.

17. The nanoparticle of claim 15 or 16, wherein the mRNAs encode a protein.

18. The composition of claim 17, wherein the protein is selected from the group consisting of: a tumor antigen, a cytokine, or a co-stimulatory molecule.

19. The nanoparticle of claim 17, wherein the protein is not expressed by a tumor cell or by a human.

20. The nanoparticle of claim 14, wherein the RNA molecules are antisense molecules, optionally siRNA, shRNA, miRNA, or any combination thereof.

21. The nanoparticle of claim 14, comprising a mixture of RNA molecules.

22. The nanoparticle of claim 21, wherein the mixture of RNA molecules is RNA isolated from cells from a human.

23. The nanoparticle of claim 22, wherein the human has a tumor and the mixture of RNA is RNA isolated from the tumor of the human, optionally, wherein the tumor is a malignant brain tumor, optionally, a glioblastoma, medulloblastoma, diffuse intrinsic pontine glioma, or a peripheral tumor with metastatic infiltration into the central nervous system.

24. The nanoparticle of any one of the preceding claims, wherein the liposomes are prepared by mixing the nucleic acid molecules and the cationic lipid at a RNA:cationic lipid ratio of about 1 to about 5 to about 1 to about 20, optionally, about 1 to about 15.

25. The nanoparticle of any one of the preceding claims, wherein the core comprises a therapeutic agent or diagnostic agent or a combination thereof.

26. The nanoparticle of claim 25, wherein the therapeutic agent is a chemotherapeutic agent or an immunotherapeutic agent.

27. The nanoparticle of claim 26, wherein the immunotherapeutic agent is a PD-L1 or PD-1 inhibitor.

28. The nanoparticle of claim 27, wherein the PD-L1 or PD-1 inhibitor is an antisense oligonucleotide or an siRNA.

29. The nanoparticle of claim 25, wherein the diagnostic agent is an imaging agent.

30. The nanoparticle of claim 29, wherein the imaging agent comprises iron oxide nanoparticles.

31. A method of making a nanoparticle comprising a positively-charged surface and an interior comprising (i) a core and (ii) at least two nucleic acid layers, wherein each nucleic acid layer is positioned between a cationic lipid bilayer, said method comprising:
(A) mixing nucleic acid molecules and liposomes at a RNA:liposome ratio of about 1 to about 5 to about 1 to about 20, optionally, about 1 to about 15, to obtain a RNA-coated liposomes, wherein the liposomes are made by a process of making liposomes comprising drying a lipid mixture comprising a cationic lipid and an organic solvent by evaporating the organic solvent under a vacuum; and
(B) mixing the RNA-coated liposomes with a surplus amount of liposomes.

32. The method of claim 31, wherein the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent.

33. The method of claim 31 or 32, wherein the process of making liposomes further comprises rehydrating the lipid mixture with a rehydration solution to form a rehydrated lipid mixture and then agitating, resting, and sizing the rehydrated lipid mixture.

34. The method of claim 33, wherein sizing the rehydrated lipid mixture comprises sonicating, extruding and/or filtering the rehydrated lipid mixture.

35. The method of any one of claims 31 to 34, comprising the steps of Example 1.

36. The method of any one of claims 31 to 35, wherein the nanoparticle has a zeta potential of about 40 mV to about 60 mV, optionally, about 45 mV to about 55 mV.

37. The method of any one of claims 31 to 36, wherein the core of the nanoparticle comprises less than about 0.5 wt % nucleic acid and/or the core comprises a cationic lipid bilayer

38. The method of any one of claims 31 to 37, wherein the outermost layer of the nanoparticle comprises a cationic lipid bilayer and/or the surface of the nanoparticle comprises a plurality of hydrophilic moieties of the cationic lipid of the cationic lipid bilayer.

39. A nanoparticle made by the method of any one of claims 31 to 39.

40. A cell comprising a nanoparticle as described in any one of claims 1 to 24 or according to claim 39.

41. The cell of claim 40, which is an antigen presenting cell (APC), optionally, a dendritic cell (DC).

42. A population of cells, wherein at least 50% of the population are cells according to claim 40 or 41.

43. A pharmaceutical composition comprising a plurality of nanoparticles according to any one of claims 1 to 24 or claim 39 and a pharmaceutically acceptable carrier, diluent, or excipient.

44. The pharmaceutical composition of claim 43, wherein the composition comprises about 10¹⁰ nanoparticles per mL to about 10¹⁵ nanoparticles per mL, optionally about 10¹² nanoparticles ±10% per mL.

45. A method of increasing an immune response against a tumor in a subject, comprising administering to the subject the pharmaceutical composition of claim 43 or 44.

46. The method of claim 45, wherein the nucleic acid molecules are mRNA.

47. The method of claim 45 or 46, wherein the composition is systemically administered to the subject.

48. The method of claim 48, wherein the composition is administered intravenously.

49. The method of any one of claims 45-48, wherein the pharmaceutical composition is administered in an amount which is effective to activate dendritic cells (DCs) in the subject.

50. The method of any one of claims 45-49, wherein the immune response is a T cell-mediated immune response.

51. The method of claim 50, wherein the T cell-mediated immune response comprises activity by tumor infiltrating lymphocytes (TILs).

52. A method of delivering RNA molecules to an intra-tumoral microenvironment, lymph node, and/or a reticuloendothelial organ, comprising administering to the subject a pharmaceutical composition of claim 43 or 44.

53. The method of claim 52, wherein the reticuloendothelial organ is a spleen or liver.

54. A method of treating a subject with a disease, comprising delivering RNA molecules to cells of the subject according to the method of claim 52 or 53.

55. The method of claim 54, wherein RNA molecules are ex vivo delivered to the cells and the cells are administered to the subject.

56. A method of treating a subject with a disease, comprising administering to the subject a pharmaceutical composition of claim 43 or 44 in an amount effective to treat the disease in the subject.

57. The method of claim 56, wherein the subject has a cancer or a tumor.

58. The method of claim 57, wherein the tumor is a malignant brain tumor, optionally, a glioblastoma, medulloblastoma, diffuse intrinsic pontine glioma, or a peripheral tumor with metastatic infiltration into the central nervous system.

59. A cell comprising a nanoparticle as described in any one of claims 25 to 30.

60. The cell of claim 59, which is an antigen presenting cell (APC), optionally, a dendritic cell (DC).

61. A population of cells, wherein at least 50% of the population are cells according to claim 59 or 60.

62. A pharmaceutical composition comprising a plurality of nanoparticles according to any one of claims 25 to 30 and a pharmaceutically acceptable carrier, diluent, or excipient.

63. The pharmaceutical composition of claim 62, wherein the composition comprises about 10¹⁰ nanoparticles per mL to about 10¹⁵ nanoparticles per mL, optionally about 10¹² nanoparticles ±10% per mL.

64. A method of increasing an immune response against a tumor in a subject, comprising administering to the subject the pharmaceutical composition of claim 62 or 63.

65. The method of claim 64, wherein the nucleic acid molecules are mRNA.

66. The method of claim 64 or 65, wherein the composition is systemically administered to the subject.

67. The method of claim 66, wherein the composition is administered intravenously.

68. The method of any one of claims 64-67, wherein the pharmaceutical composition is administered in an amount which is effective to activate dendritic cells (DCs) in the subject.

69. The method of any one of claims 64-68, wherein the immune response is a T cell-mediated immune response.

70. The method of claim 69, wherein the T cell-mediated immune response comprises activity by tumor infiltrating lymphocytes (TILs).

71. A method of delivering RNA molecules to an intra-tumoral microenvironment, lymph node, and/or a reticuloendothelial organ, comprising administering to the subject a pharmaceutical composition of claim 43 or 44.

72. The method of claim 52, wherein the reticuloendothelial organ is a spleen or liver.

73. A method of treating a subject with a disease, comprising delivering RNA molecules to cells of the subject according to the method of claim 52 or 53.

74. The method of claim 54, wherein RNA molecules are ex vivo delivered to the cells and the cells are administered to the subject.

75. A method of treating a subject with a disease, comprising administering to the subject a pharmaceutical composition of claim 62 or 63 in an amount effective to treat the disease in the subject.

76. The method of claim 75, wherein the subject has a cancer or a tumor.

77. The method of claim 76, wherein the tumor is a malignant brain tumor, optionally, a glioblastoma, medulloblastoma, diffuse intrinsic pontine glioma, or a peripheral tumor with metastatic infiltration into the central nervous system.
